SEQUENCE LISTING (6 sequences)

SEQ ID NO: 1 - DNA, 4586 bp, Homo sapiens
  CDS (146)...(2368)
ccacgcgtccgattccagccatccctctgcctgcaatgagagcttcccgccgcctcagccacagtcccacccgggggccttgggccccagacatgcggtgatctcagggcaagggttgccacgaccacccagaacctcaccagccatgaaagcccaccccaaggagatggtgcctctcatgggcaagagagttgctgcccccagtgggaaccctgccgtcctgccagagaagaggccggcggagatcacccccacaaagaagagtgcacacttcttcctggagatagaagggtttgaacccaaccccacagttgccaagacctctcctcctgtcttctccaagcccatggattccaacatccggcagtgcatctctggtaactgtgatgacatggactccccccagtctcctcaagatgatgtgacagagaccccatccaatcccaacagccccagtgcacagctggccaaggaagagcagaggaggaaaaagaggcggctgaagaagcgcatctttgcagccgtgtctgagggctgcgtggaggagttggtagagttgctggtggagctgcaggagctttgcaggcggcgccatgatgaggatgtgcctgacttcctcatgcacaagctgacggcctccgacacggggaagacctgcctgatgaaggccttgttaaacatcaaccccaacaccaaggagatcgtgcggatcctgcttgcctttgctgaagagaacgacatcctgggcaggttcatcaacgccgagtacacagaggaggcctatgaagggcagacggcgctgaacatcgccatcgagcggcggcagggggacatcgcagccctgctcatcgccgccggcgccgacgtcaacgcgcacgccaagggggccttcttcaaccccaagtaccaacacgaaggcttctacttcggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgctgcagctggccgccaagatgggcaaggcggagatcctgaagtacatcctcagtcgtgagatcaaggagaagcggctccggagcctgtccaggaagttcaccgactgggcgtacggacccgtgtcatcctccctctacgacctcaccaacgtggacaccaccacggacaactcagtgctggaaatcactgtctacaacaccaacatcgacaaccggcatgagatgctgaccctggagccgctgcacacgctgctgcatatgaagtggaagaagtttgccaagcacatgttctttctgtccttctgcttttatttcttctacaacatcaccctgaccctcgtctcgtactaccgcccccgggaggaggaggccatcccgcaccccttggccctgacgcacaagatggggtggctgcagctcctagggaggatgtttgtgctcatctgggccatgtgcatctctgtgaaagagggcattgccatcttcctgctgagaccctcggatctgcagtccatcctctcggatgcctggttccactttgtcttttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtcattttgcatgatgttctgaagttcttgtttgtatatatcgtgtttttgcttggatttggagtagccttggcctcgctgatcgagaagtgtcccaaagacaacaaggactgcagctcctacggcagcttcagtgacgcagtgctggaactcttcaagctcaccataggcctgggtgatctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgcagagagccaggaccatcttggagtttgagaaaatgttaccagaatggctgaggagcagattccggatgggagagctgtgcaaagtggccgaggatgatttccgactgtgtttgcggatcaatgaggtgaagtggactgaatggaagacgcacgtctccttccttaacgaagacccggggcctgtaagacgaacagatttcaacaaaatccaagattcttccaggaacaacagcaaaaccactctcaatgcatttgaagaagtcgaggaattcccggaaacctcggtgtagaagcggaacccagagctggtgtgcgcgtgcgctgtctggcgctgcaggcggagtcaccgactctgtgcagagaggctttgagggatgatggagtccggctctgctggcctagaagcagagtgcaccctcgtgctcagtgctcagtgggtgtctgaactgaggggcagttgtcaatttgtctgagtgggaaacatcctggattttgttacttggcaaacagctggtgtaaacctacagccagcagcagtctggagcctgggagcctcctgaagtcccgggtgaagcctctggttttaccaattgcaggtcggcttggctgggagagatggatggcgggaaaggggcagcagtcttgaggagcagggagaggagtctttcctcctgccagcttcccccgtcagccccaaccccagcccacacattgtaccatctcttctgctgtgactgggttgcctgaatttgtgggagacccgtgatcccatcccagagtgtgcgggggacggaggtaagctggatatcctgggggaggaggggaatgcgctctggaaacacccttccggaacccttcggggaaaaggagaccatccttggagtgaacgtcccctgacaccccaaggttcaaactgtctcaagctgagagatgtttttagtagcataattaacacagggttttaacttgcaatacggaaaagacatttcagttgagaatgaaaattactacaatgaagtttgtgattttaaaagtggagacagactgggggctttggggctggatgtaagtattatatatttggcctcagggtgcccagagcaagacaaaaagcttttcttcacacacacaaaagtctgcatgagacactccgggcaagtcctgctgggccgccgcgatctgggtgaaaggtcctggctcttttcctcgtcctgacctcacagtagcgcatgcctgtgtgctgggatcgtggctttcgctgaagcagaaatagcagctgctcgatcgatatcatcttggaactcagcagttagtcgcatacctcagtacgtctcagtgggggaatttaacaaaatgcctcaactgctttggtacgaagtattttttttttaattttaactgtgaattttgaagctgaaggggaagcttgtgagagaaaagcatttgccaagactttgagcttatttttaggtcctcgtcctctgatgttctctttctgaaatgacacggagtcagtctcgggggcagaggtgaagtggagacggaaggattttccaggtgactggggccgaaaccaccagaaaatccactctgccgccgttatctggtgaaaggattcatgtaaaaatgttcgaggtggaattataaaaatagtaaccataaatgttaatcttaaatggcagaaatagaaatttggccttcagataacatggcgatagataagttcatctggcttgaggcaaactgaagagtcggggcctagcattgcactctgggccagtttctctgccctgggccactctgtgtgccagactagctggacagatagagactttgtgcccctgatggggccgattggggagaggtgggctggggtgtgcaggcttcacaatccacagcagcccctgccctcccagctgacccagggagtaatcgcgtgctctaagccacagtggtcggggctgggcatgggcctctggagaagagaagatttgaggagaactgtcctagaggcaggaggagcagatgtgtttcagaatgggcagaattaggaaattgagaaagattttggctcaacagaatccagcaactgctccagatgttggagatgtttaagcagaagctggttgagcacttaatgaggaatgttgttgaaaatggtcattggaagaagtttaaggtcccttttagcctggagattgtacaaatcagcattccacatctggagttagctacccgcattaagcctgaacagacatcttggtctgaaaggaagtggtttggattcatgatgccaagctccacactatggagctgggaattccagaattgctttgactcagatattaatggagaaag

SEQ ID NO: 2 - protein, 741 aa, Homo sapiens
MKAHPKEMVPLMGKRVAAPSGNPAVLPEKRPAEITPTKKSAHFFLEIEGFEPNPTVAKTSPPVFSKPMDSNIRQCISGNCDDMDSPQSPQDDVTETPSNPNSPSAQLAKEEQRRKKRRLKKRIFAAVSEGCVEELVELLVELQELCRRRHDEDVPDFLMHKLTASDTGKTCLMKALLNINPNTKEIVRILLAFAEENDILGRFINAEYTEEAYEGQTALNIAIERRQGDIAALLIAAGADVNAHAKGAFFNPKYQHEGFYFGETPLALAACTNQPEIVQLLMEHEQTDITSRDSRGNNILHALVTVAEDFKTQNDFVKRMYDMILLRSGNWELETTRNNDGLTPLQLAAKMGKAEILKYILSREIKEKRLRSLSRKFTDWAYGPVSSSLYDLTNVDTTTDNSVLEITVYNTNIDNRHEMLTLEPLHTLLHMKWKKFAKHMFFLSFCFYFFYNITLTLVSYYRPREEEAIPHPLALTHKMGWLQLLGRMFVLIWAMCISVKEGIAIFLLRPSDLQSILSDAWFHFVFFIQAVLVILSVFLYLFAYKEYLACLVLAMALGWANMLYYTRGFQSMGMYSVMIQKVILHDVLKFLFVYIVFLLGFGVALASLIEKCPKDNKDCSSYGSFSDAVLELFKLTIGLGDLNIQQNSKYPILFLFLLITYVILTFVLLLNMLIALMGETVENVSKESERIWRLQRARTILEFEKMLPEWLRSRFRMGELCKVAEDDFRLCLRINEVKWTE

SEQ ID NO: 3 - DNA, 2223 bp, Homo sapiens
  CDS (1)...(2223)
atgaaagcccaccccaaggagatggtgcctctcatgggcaagagagttgctgcccccagtgggaaccctgccgtcctgccagagaagaggccggcggagatcacccccacaaagaagagtgcacacttcttcctggagatagaagggtttgaacccaaccccacagttgccaagacctctcctcctgtcttctccaagcccatggattccaacatccggcagtgcatctctggtaactgtgatgacatggactccccccagtctcctcaagatgatgtgacagagaccccatccaatcccaacagccccagtgcacagctggccaaggaagagcagaggaggaaaaagaggcggctgaagaagcgcatctttgcagccgtgtctgagggctgcgtggaggagttggtagagttgctggtggagctgcaggagctttgcaggcggcgccatgatgaggatgtgcctgacttcctcatgcacaagctgacggcctccgacacggggaagacctgcctgatgaaggccttgttaaacatcaaccccaacaccaaggagatcgtgcggatcctgcttgcctttgctgaagagaacgacatcctgggcaggttcatcaacgccgagtacacagaggaggcctatgaagggcagacggcgctgaacatcgccatcgagcggcggcagggggacatcgcagccctgctcatcgccgccggcgccgacgtcaacgcgcacgccaagggggccttcttcaaccccaagtaccaacacgaaggcttctacttcggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgctgcagctggccgccaagatgggcaaggcggagatcctgaagtacatcctcagtcgtgagatcaaggagaagcggctccggagcctgtccaggaagttcaccgactgggcgtacggacccgtgtcatcctccctctacgacctcaccaacgtggacaccaccacggacaactcagtgctggaaatcactgtctacaacaccaacatcgacaaccggcatgagatgctgaccctggagccgctgcacacgctgctgcatatgaagtggaagaagtttgccaagcacatgttctttctgtccttctgcttttatttcttctacaacatcaccctgaccctcgtctcgtactaccgcccccgggaggaggaggccatcccgcaccccttggccctgacgcacaagatggggtggctgcagctcctagggaggatgtttgtgctcatctgggccatgtgcatctctgtgaaagagggcattgccatcttcctgctgagaccctcggatctgcagtccatcctctcggatgcctggttccactttgtcttttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtcattttgcatgatgttctgaagttcttgtttgtatatatcgtgtttttgcttggatttggagtagccttggcctcgctgatcgagaagtgtcccaaagacaacaaggactgcagctcctacggcagcttcagtgacgcagtgctggaactcttcaagctcaccataggcctgggtgatctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgcagagagccaggaccatcttggagtttgagaaaatgttaccagaatggctgaggagcagattccggatgggagagctgtgcaaagtggccgaggatgatttccgactgtgtttgcggatcaatgaggtgaagtggactgaa

SEQ ID NO: 4 - DNA, 3042 bp, Homo sapiens
  CDS (1)...(3039)
atggttggaggatgcaggtggacagaagacgtggagcctgcagaagtaaaggaaaagatgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttaccaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacctcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagaagagcaaaccgaggctcagagacacaataatccaggtcacatggctggaaaatggtagaatcaaggttgagagcaaagatgtgactgacggcaaagcctcttctcatatgctggtggttctcaagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacctgacccgggctgttggtgagtccacagtgtggaatgctgtggtgggcgcggatctgccatgtggcacagacattgccagcggcactcatagaccagatggtggagagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagggccggccggctgctcccagtgcggggcccgccaagcccacgcccacccggaactccatctggcccgcaagctccacacgcagccccggttcccgctcacgccactccttccacacttccctgcaagctgagggtgccagctctggccttggccagcccagaaaggggctcccacagtgcagcggtgggctgaagggctcctcaagtgccgccaaagtgggagcccaggcagaggaggtgccgagagcaagcgagggctgtgaggactgccagcacgctgtcacctctcagaagcgtaagacagcaatggaccaaacagacgaagatctcttcccctatggagcattctaccagttcctgatgatttccaggagctttcgaggagaggagatgagcatcggcaagcagcactaa

SEQ ID NO: 5 - protein, 1013 aa, Homo sapiens [sequence omitted]

SEQ ID NO: 6 - DNA, 3039 bp, Homo sapiens
  CDS (1)...(3039)
atggttggaggatgcaggtggacagaagacgtggagcctgcagaagtaaaggaaaagatgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttaccaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacctcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagaagagcaaaccgaggctcagagacacaataatccaggtcacatggctggaaaatggtagaatcaaggttgagagcaaagatgtgactgacggcaaagcctcttctcatatgctggtggttctcaagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacctgacccgggctgttggtgagtccacagtgtggaatgctgtggtgggcgcggatctgccatgtggcacagacattgccagcggcactcatagaccagatggtggagagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagggccggccggctgctcccagtgcggggcccgccaagcccacgcccacccggaactccatctggcccgcaagctccacacgcagccccggttcccgctcacgccactccttccacacttccctgcaagctgagggtgccagctctggccttggccagcccagaaaggggctcccacagtgcagcggtgggctgaagggctcctcaagtgccgccaaagtgggagcccaggcagaggaggtgccgagagcaagcgagggctgtgaggactgccagcacgctgtcacctctcagaagcgtaagacagcaatggaccaaacagacgaagatctcttcccctatggagcattctaccagttcctgatgatttccaggagctttcgaggagaggagatgagcatcggcaagcagcac

(19) United States
(12) Patent Application Publication
(10) Pub. No.: US 2002/0156253 A1
(43) Pub. Date: Oct. 24, 2002

(54) 48000 and 52920, novel human calcium channels and uses thereof

(76) Inventors: Rory A.J. Curtis, Southborough, MA (US); Inmaculada Silos-Santiago, Cambridge, MA (US)

Correspondence Address: LAHIVE & COCKFIELD, 28 STATE STREET, BOSTON, MA, 02109, US

(21) Appl. No.: 09/891,762

(22) Filed: Jun. 26, 2001

Related U.S. Application Data
(60) Provisional application No. 60/214,176, filed on Jun. 26, 2000.

(51) Int. Cl. C07H 21/02; C07H 21/04

(52) U.S. Cl. 536/23.1

(57) ABSTRACT

The invention provides isolated nucleic acids molecules, designated TLCC-4 and TLCC-5 nucleic acid molecules, which encode novel TRP-like calcium channel molecules. The invention also provides antisense nucleic acid molecules, recombinant expression vectors containing TLCC-4 or TLCC-5 nucleic acid molecules, host cells into which the expression vectors have been introduced, and nonhuman transgenic animals in which a TLCC-4 or TLCC-5 gene has been introduced or disrupted. The invention still further provides isolated TLCC-4 or TLCC-5 polypeptides, fusion polypeptides, antigenic peptides and anti-TLCC-4 or anti-TLCC-5 antibodies. Diagnostic methods utilizing compositions of the invention are also provided.

### RELATED APPLICATIONS

This application claims priority to U.S. Provisional Patent Application No. 60/214,176 filed on Jun. 26, 2000, incorporated herein in its entirety by reference.

### BACKGROUND OF THE INVENTION

Calcium signaling has been implicated in the regulation of a variety of cellular responses, such as growth and differentiation. There are two general methods by which intracellular concentrations of calcium ions may be increased: calcium ions may be freed from intracellular stores, transported by specific membrane channels in the storage organelle, or calcium ions may be brought into the cell from the extracellular milieu through the use of specific channels in the cellular membrane. In the situation in which the intracellular stores of calcium have been depleted, a specific type of calcium channel, termed a ‘capacitative calcium channel’ or a ‘store-operated calcium channel’ (SOC), is activated in the plasma membrane to import calcium ions from the extracellular environment to the cytosol (see Putney and McKay (1999) BioEssays 21:38-46). Calcium may also enter the cell via receptor-stimulated cation channels (see Hofmann et al. (2000) J. Mol. Med. 78:14-25).

There is no single electrophysological profile characteristic of the calcium channel family; rather, a wide array of single channel conductances, cation selectivity, and current properties have been observed for different channels. Further, in several instances it has been demonstrated that homo- or hetero-polymerization of the channel molecule may occur, further changing the channel properties from those of the single molecule. In general, though, these channels function similarly, in that they are calcium ion-permeable cation channels which become activated after agonist binding to a G protein-coupled receptor.

Members of the capacitative calcium channel family include the calcium release-activated calcium current (CRAC) (Hoth and Penner (1992) Nature 355: 353-355), calcium release-activated non-selective cation current (CRANC) (Krause et al. (1996) J. Biol. Chem. 271: 32523-32528), and the transient receptor potential (TRP) proteins TRP1, TRP2, TRP4, and TRP5. Depletion of intracellular calcium stores activate these channels by a mechanism which is yet undefined, but which has been demonstrated to involve a diffusible factor using studies in which calcium stores were artificially depleted (e.g., by the introduction of chelators into the cell, by activating phospholipase C_{γ}, or by inhibiting those enzymes responsible for pumping calcium ions into the stores or those enzymes responsible for maintaining resting intracellular calcium ion concentrations) (Putney, J. W., (1986) Cell Calcium 7:1-12; Putney, J. W. (1990) Cell Calcium 11:611-624).

Recently, it has been elucidated that three TRP family members, TRP3, TRP6, and a mouse homologue, TRP7, form a sub-family of receptors that are activated in a calcium store-depletion independent manner. TRP3 and TRP6 are activated by diacylglycerols in a membrane delimited manner (Hofmann et al. (1999) Nature 397:259-263). Similarly, murine TRP7 is activated via diacylglycerol stimulation by G_{q} protein coupled receptors (Okada et al. (1999) J. Biol. Chem. 274:27359-27370).

The TRP channel family is one of the best characterized calcium channel protein families. These channels include transient receptor potential protein and homologues thereof (to date, seven TRP homologues and splice variants have been identified in a variety of organisms), the vanilloid receptor subtype I (also known as the capsaicin receptor); the stretch-inhibitable non-selective cation channel (SIC); the olfactory, mechanosensitive channel; the insulin-like growth factor I-regulated calcium channel; the vitamin D-responsive apical, epithelial calcium channel (ECaC); melastatin; and the polycystic kidney disease protein family (see, e.g., Montell and Rubin (1989) Neuron 2:1313-1323; Caterina et al. (1997) Nature 389: 816-824; Suzuki et al. (1999) J. Biol. Chem. 274: 6330-6335; Kiselyov et al. (1998) Nature 396: 478-482; Hoenderop et al. (1999) J. Biol. Chem. 274: 8375-8378; and Chen et al. (1999) Nature 401(6751): 383-6). Each of these molecules is 700 or more amino acids in length, and shares certain conserved structural features. Predominant among these structural features are six transmembrane domains, with an additional hydrophobic loop present between the fifth and sixth transmembrane domains. It is believed that this loop is integral to the activity of the pore of the channel formed upon membrane insertion (Hardie and Minke (1993) Trends Neurosci 16: 371-376). TRP channel proteins also include one or more ankyrin domains. Although found in disparate tissues and organisms, members of the TRP channel protein family all serve to transduce signals by means of calcium entry into cells, particularly pain signals (see, e.g., McClesky and Gold (1999) Annu. Rev. Physiol. 61: 835-856), light signals (Hardie and Minke, supra), or olfactory signals (Colbert et al. (1997) J. Neurosci 17(21): 8259-8269). Thus, this family of molecules may play important roles in sensory signal transduction in general.

Vanilloid receptors (VRs) are cation channels that are structurally related to members of the TRP family of ion channels. VRs share several physical characteristics including an N-terminal cytoplasmic domain which contains three ankyrin repeats, six transmembrane domains, a pore-loop region located between transmembrane domains 5 and 6, and several kinase consensus sequences. These receptors have been proposed to mediate the entry of extracellular calcium into cells in response to the depletion of intracellular calcium stores. VRs are expressed in nociceptive neurons, as well as other cells types, and are activated by a variety of stimuli including noxious heat and protons. A well-known agonist of VR1 is capsaicin, which induces pain behavior in humans and rodents. VR1 knockout mice have been shown to be impaired in their detection of painful heat, to exhibit no vanilloid-evoked pain behavior, and to show little thermal hypersensitivity after inflammation (Szallasi and Blumberg (1999) Pharmacol. Rev. 51:159-211).

### SUMMARY OF THE INVENTION

The present invention is based, at least in part, on the discovery of novel calcium channel family members, referred to herein as “TRP-like calcium channel 4” and “TRP-like calcium channel-5” or “TLCC-4” and “TLCC-5” nucleic acid and polypeptide molcules. The TLCC-4 and TLCC-5 nucleic acid and polypeptide molecules of the present invention are useful as modulating agents in regulating a variety of cellular processes, e.g., membrane excitability, neurite outgrowth and synaptogenesis, signal transduction, cell proliferation, growth, differentiation, and migration, and nociception. Accordingly, in one aspect, this invention provides isolated nucleic acid molecules encoding TLCC-4 and TLCC-5 polypeptides or biologically active portions thereof, as well as nucleic acid fragments suitable as primers or hybridization probes for the detection of TLCC-4-encoding and TLCC-5-encoding nucleic acids.

In one embodiment, the invention features an isolated nucleic acid molecule that includes the nucleotide sequence set forth in SEQ ID NO:1, 3, 4, or 6. In another embodiment, the invention features an isolated nucleic acid molecule that encodes a polypeptide including the amino acid sequence set forth in SEQ ID NO:2 or 5. In another embodiment, the invention features isolated nucleic acid molecules that include the nucleotide sequences contained in the plasmids deposited with ATCC® as Accession Numbers______.

In still other embodiments, the invention features isolated nucleic acid molecules including nucleotide sequences that are substantially identical (e.g., 60%, 65%, 70%, 75%, 80%, 85%, 90%, 95% or 98% identical) to the nucleotide sequence set forth as SEQ ID NO:1, 3, 4, or 6. The invention further features isolated nucleic acid molecules including at least 50 contiguous nucleotides of the nucleotide sequence set forth as SEQ ID NO:1, 3, 4, or 6. In another embodiment, the invention features isolated nucleic acid molecules which encode a polypeptide including an amino acid sequence that is substantially identical (e.g., 60%, 65%, 70%, 75%, 80%, 85%, 90%, 95% or 98% identical) to the amino acid sequence set forth as SEQ ID NO:2 or 5. The present invention also features nucleic acid molecules which encode allelic variants of the polypeptide having the amino acid sequence set forth as SEQ ID NO:2 or 5. In addition to isolated nucleic acid molecules encoding full-length polypeptides, the present invention also features nucleic acid molecules which encode fragments, for example biologically active or antigenic fragments, of the full-length polypeptides of the present invention (e.g., fragments including at least 10, 15, 20, 25, 30, 35, 40, 45 or 50 contiguous amino acid residues of the amino acid sequence of SEQ ID NO:2 or 5). In still other embodiments, the invention features nucleic acid molecules that are complementary to, antisense to, or hybridize under stringent conditions to the isolated nucleic acid molecules described herein.

In another aspect, the invention provides vectors including the isolated nucleic acid molecules described herein (e.g., TLCC-4-encoding or TLCC-5-encoding nucleic acid molecules). Such vectors can optionally include nucleotide sequences encoding heterologous polypeptides. Also featured are host cells including such vectors (e.g., host cells including vectors suitable for producing TLCC-4 or TLCC-5 nucleic acid molecules and polypeptides).

In another aspect, the invention features isolated TLCC-4 or TLCC-5 polypeptides and/or biologically active or antigenic fragments thereof. Exemplary embodiments feature a polypeptide including the amino acid sequence set forth as SEQ ID NO:2 or 5, a polypeptide including an amino acid sequence at least 60%, 65%, 70%, 75%, 80%, 85%, 90%, 95% or 98% identical to the amino acid sequence set forth as SEQ ID NO:2 or 5, a polypeptide encoded by a nucleic acid molecule including a nucleotide sequence at least 60%, 65%, 70%, 75%, 80%, 85%, 90%, 95% or 98% identical to the nucleotide sequence set forth as SEQ ID NO:1, 3, 4, or 6. Also featured are fragments of the full-length polypeptides described herein (e.g., fragments including at least 10 contiguous amino acid residues of the sequence set forth as SEQ ID NO:2 or 5) as well as allelic variants of the polypeptide having the amino acid sequence set forth as SEQ ID NO:2 or 5.

The TLCC-4 or TLCC-5 polypeptides and/or biologically active or antigenic fragments thereof, are useful, for example, as reagents or targets in assays applicable to treatment and/or diagnosis of TLCC-4 or TLCC-5 mediated or related disorders. In one embodiment, a TLCC-4 or TLCC-5 polypeptide, or fragment thereof, has a TLCC-4 or TLCC-5 activity. In another embodiment, a TLCC-4 or TLCC-5 polypeptide, or fragment thereof, has one or more of the following domains: an ankyrin repeat domain, a transmembrane domain, a pore domain, a transient receptor domain, a kinase consensus sequence, and/or an ion transport protein domain, and optionally, has a TLCC-4 or TLCC-5 activity. In a related aspect, the invention features antibodies (e.g., antibodies which specifically bind to any one of the polypeptides described herein) as well as fusion polypeptides including all or a fragment of a polypeptide described herein.

The present invention further features methods for detecting TLCC-4 or TLCC-5 polypeptides and/or TLCC-4 or TLCC-5 nucleic acid molecules, such methods featuring, for example, a probe, primer or antibody described herein. Also featured are kits, e.g., kits for the detection of TLCC-4 or TLCC-5 polypeptides and/or TLCC-4 or TLCC-5 nucleic acid molecules. In a related aspect, the invention features methods for identifying compounds which bind to and/or modulate the activity of a TLCC-4 or TLCC-5 polypeptide or TLCC-4 or TLCC-5 nucleic acid molecule described herein. Further featured are methods for modulating a TLCC-4 or TLCC-5 activity.

Other features and advantages of the invention will be apparent from the following detailed description and claims.

### BRIEF DESCRIPTION OF THE DRAWINGS

FIG. 1 depicts the cDNA sequence and predicted amino acid sequence of human TLCC-4. The nucleotide sequence corresponds to nucleic acids 1 to 4586 of SEQ ID NO:1. The amino acid sequence corresponds to amino acids 1 to 742 of SEQ ID NO:2. The coding region without the 5′ and 3′ untranslated regions of the human TLCC-4 gene is shown in SEQ ID NO:3.

FIG. 2 depicts the cDNA sequence and predicted amino acid sequence of human TLCC-5. The nucleotide sequence corresponds to nucleic acids 1 to 3042 of SEQ ID NO:4. The amino acid sequence corresponds to amino acids 1 to 1013 of SEQ ID NO:5. The coding region of the human TLCC-5 gene is shown in SEQ ID NO:6.

FIG. 3 depicts TLCC-4 mRNA expression in normal human tissues, as determined using RT-PCR.

FIG. 4 depicts a structural, hydrophobicity, and antigenicity analysis of the human TLCC-4 polypeptide (SEQ ID NO:2).

FIG. 5 depicts the results of a search which was performed against the HMM database in PFAM and which resulted in the identification of three “ankyrin repeat domains” and an “ion transport protein domain” in the human TLCC-4 polypeptide (SEQ ID NO:2).

FIG. 6 depicts the results of a search which was performed against the MEMSAT database and which resulted in the identification of six “transmembrane domains” in the human TLCC-4 polypeptide (SEQ ID NO:2).

FIG. 7 depicts a structural, hydrophobicity, and antigenicity analysis of the human TLCC-5 polypeptide (SEQ ID NO:5).

FIG. 8 depicts the results of a search which was performed against the HMM database which resulted in the identification of two “transient receptor domains” in the amino acid sequence of the human TLCC-5 polypeptide (SEQ ID NO:5).

FIG. 9 depicts the results of a search which was performed against the MEMSAT database and which resulted in the identification of two “transmembrane domains” in the human TLCC-5 polypeptide (SEQ ID NO:5).

FIG. 10 depicts an alignment of the human TLCC-5 amino acid sequence (SEQ ID NO:5) with the amino acid sequence of human transient receptor 7 (hTRP7), using the CLUSTAL W (1.74) alignment program.

FIG. 11 depicts an alignment of the human TLCC-5 amino acid sequence (SEQ ID NO:5) with the amino acid sequence of human melastatin, using the CLUSTAL W (1.74) alignment program.

FIG. 12 is a graph depicting the expression of human TLCC-4 cDNA (SEQ ID NO:3) in various human tissues as determined by Taqman analysis.

FIG. 13 is a graph depicting the expression of human TLCC-4 cDNA (SEQ ID NO:3) in various diseased and normal state human tissues as determined by Taqman analysis.

FIG. 14 is a graph depicting the expression of human TLCC-4 cDNA (SEQ ID NO:3) in normal monkey and human tissues as determined by Taqman analysis.

FIG. 15 is a graph depicting the expression of human TLCC-4 cDNA (SEQ ID NO:3) in normal monkey and human tissues as determined by Taqman analysis.

FIG. 16 is a graph depicting the expression of human TLCC-4 cDNA (SEQ ID NO:3) in various tissues involved in metabolism as determined by Taqman analysis.

### DETAILED DESCRIPTION OF THE INVENTION

The present invention is based, at least in part, on the discovery of novel molecules, referred to herein as “TRP-like calcium channel-4” and “TRP-like calcium channel-5” or “TLCC-4” and “TLCC-5” nucleic acid and polypeptide molecules, which are novel members of the ion channel, e.g., calcium channel and/or vanilloid receptor, family. These novel molecules are capable of, for example, modulating an ion-channel mediated activity (e.g., a calcium channel- and/or vanilloid receptor-mediated activity) in a cell, e.g., a neuronal, skin, muscle (e.g., cardiac muscle), or liver cell.

As used herein, an “ion channel” includes a protein or polypeptide which is involved in receiving, conducting, and transmitting signals in an electrically excitable cell, e.g., a neuronal or muscle cell. Ion channels include calcium channels, potassium channels, and sodium channels. As used herein, a “calcium channel” includes a protein or polypeptide which is involved in receiving, conducting, and transmitting calcium ion-based signals in an electrically excitable cell. Calcium channels are calcium ion selective, and can determine membrane excitability (the ability of, for example, a neuronal cell to respond to a stimulus and to convert it into a sensory impulse). Calcium channels can also influence the resting potential of membranes, wave forms and frequencies of action potentials, and thresholds of excitation. Calcium channels are typically expressed in electrically excitable cells, e.g., neuronal cells, and may form heteromultimeric structures (e.g., composed of more than one type of subunit). Calcium channels may also be found in non-excitable cells (e.g., adipose cells or liver cells), where they may play a role in, e.g., signal transduction. Examples of calcium channels include the low-voltage-gated channels and the high-voltage-gated channels. Calcium channels are described in, for example, Davila et al. (1999) Annals New York Academy of Sciences 868:102-17 and McEnery, M. W. et al. (1998) J. Bioenergetics and Biomembranes 30(4): 409-418, the contents of which are incorporated herein by reference. As the TLCC-4 and TLCC-5 molecules of the present invention are calcium channels modulating ion channel mediated activities (e.g., calcium channel- and/or vanilloid receptor-mediated activities), they may be useful for developing novel diagnostic and therapeutic agents for ion channel associated disorders (e.g., calcium channel and/or vanilloid receptor associated disorders).

As used herein, an “ion channel associated disorder” includes a disorder, disease or condition which is characterized by a misregulation of an ion channel mediated activity. For example, a “calcium channel associated disorder” includes a disorder, disease or condition which is characterized by a misregulation of a calcium channel mediated activity. Ion channel associated disorders, e.g., calcium channel associated disorders, include CNS disorders, such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, Creutzfeldt-Jakob disease, or AIDS related dementia; autonomic function disorders such as hypertension and sleep disorders, and neuropsychiatric disorders, such as depression, schizophrenia, schizoaffective disorder, korsakoff's psychosis, mania, anxiety disorders, or phobic disorders; leaning or memory disorders, e.g., amnesia or age-related memory loss, attention deficit disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Ion channel associated disorders, e.g., vanilloid receptor associated disorders also include pain disorders. As used herein, the term “pain disorders” includes those disorders, diseases or conditions that affect pain signaling mechanisms. Examples of pain disorders include postherpetic neuralgia, diabetic neuropathy, postmastectomy pain syndrome, stump pain, reflex sympathetic dystrophy, trigeminal neuralgia, neuropathic pain, orofacial neuropathic pain, osteoarthritis, rheumatoid arthritis, fibromyalgia syndrome, tension myalgia, Guillian-Barre syndrome, Meralgia paraesthetica, burning mouth syndrome, fibrocitis, myofascial pain syndrome, idiopathic pain disorder, temporomandibular joint syndrome, atypical odontalgia, loin pain, haematuria syndrome, non-cardiac chest pain, low back pain, chronic nonspecific pain, psychogenic pain, musculoskeletal pain disorder, chronic pelvic pain, nonorganic chronic headache, tension-type headache, cluster headache, migraine, complex regional pain syndrome, vaginismus, nerve trunk pain, somatoform pain disorder, cyclical mastalgia, chronic fatigue syndrome, multiple somatization syndrome, chronic pain disorder, somatization disorder, Syndrome X, facial pain, idiopathic pain disorder, posttraumatic rheumatic pain modulation disorder (fibrositis syndrome), and Tangier disease.

As used herein, the term “pain signaling mechanisms” include the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The TLCC-4 or TLCC-5 molecules of the present invention may be present on these sensory neurons and, thus, may be involved in detecting these noxious chemical, mechanical, or thermal stimuli and transducing this information into membrane depolarization events. Thus, the TLCC-4 or TLCC-5 molecules by participating in pain signaling mechanisms, may modulate pain elicitation and act as targets for developing novel diagnostic targets and therapeutic agents to control pain.

Ion channel associated disorders, e.g., calcium channel and/or vanilloid receptor disorders, also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The TLCC-4 or TLCC-5 molecules of the present invention are involved in signal transduction mechanisms, which are known to be involved in cellular growth, differentiation, and migration processes. Thus, the TLCC-4 or TLCC-5 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; hepatic disorders; cardiovascular disorders; and hematopoietic and/or myeloproliferative disorders.

As used herein, an “ion channel mediated activity” includes an activity which involves an ion channel, e.g., an ion channel and/or a vanilloid receptor, in a neuronal cell, a muscular cell, a skin cell or a liver cell, associated with receiving, conducting, and transmitting signals. Ion channel mediated activities (e.g., calcium channel and/or vanilloid receptor mediated activities) include release of neurotransmitters or second messenger molecules (e.g., dopamine or norepinephrine), from cells, e.g., neuronal cells; modulation of resting potential of membranes, wave forms and frequencies of action potentials, and thresholds of excitation; participation in signal transduction pathways, and modulation of processes such as integration of sub-threshold synaptic responses and the conductance of back-propagating action potentials in, for example, neuronal cells (e.g., changes in those action potentials resulting in a morphological or differentiative response in the cell).

The term “family” when referring to the polypeptide and nucleic acid molecules of the invention is intended to mean two or more polypeptides or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

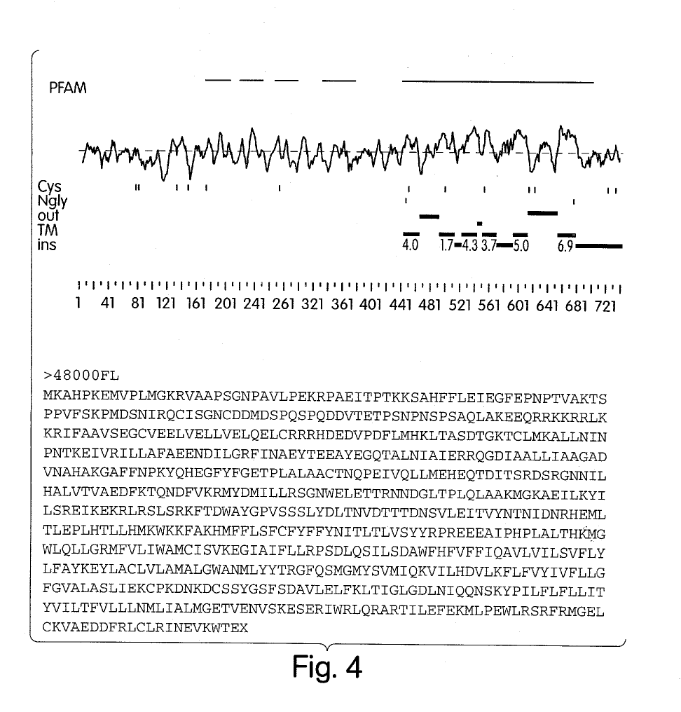

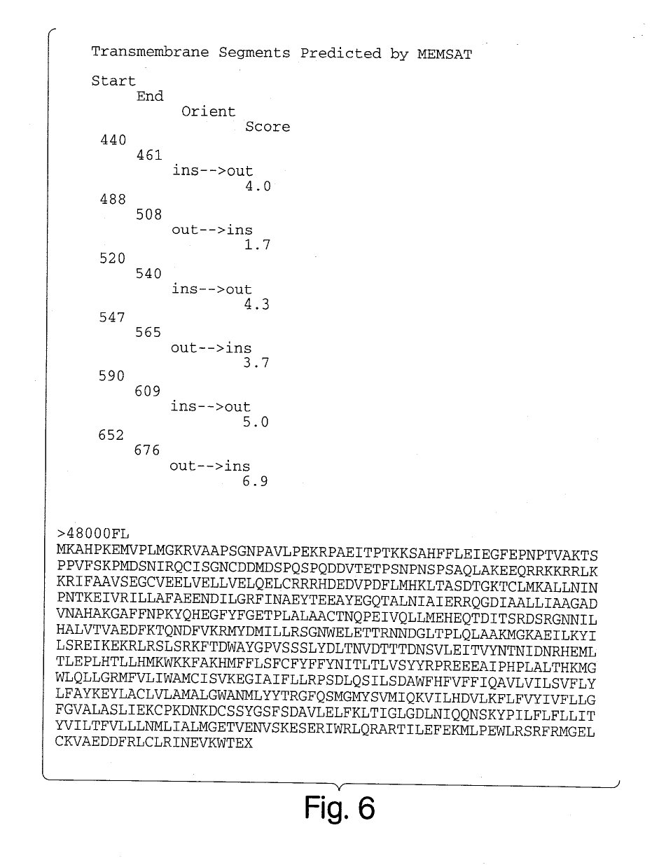

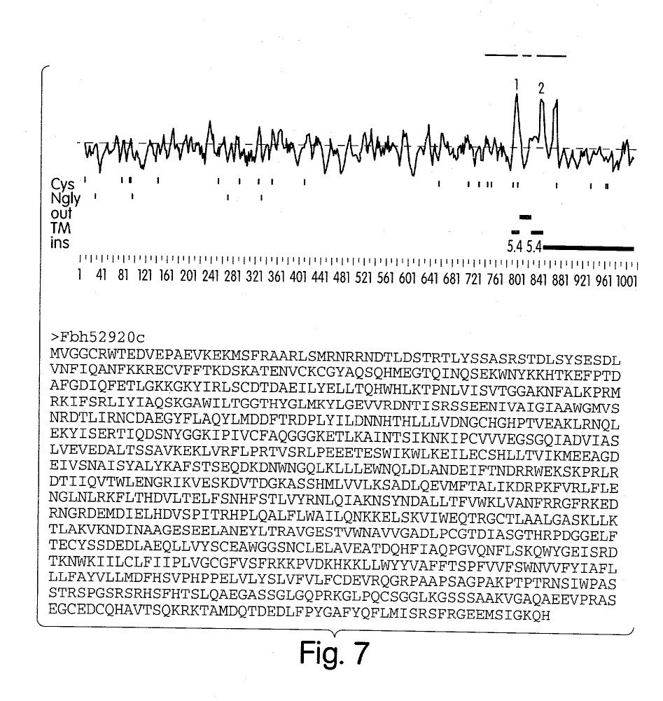

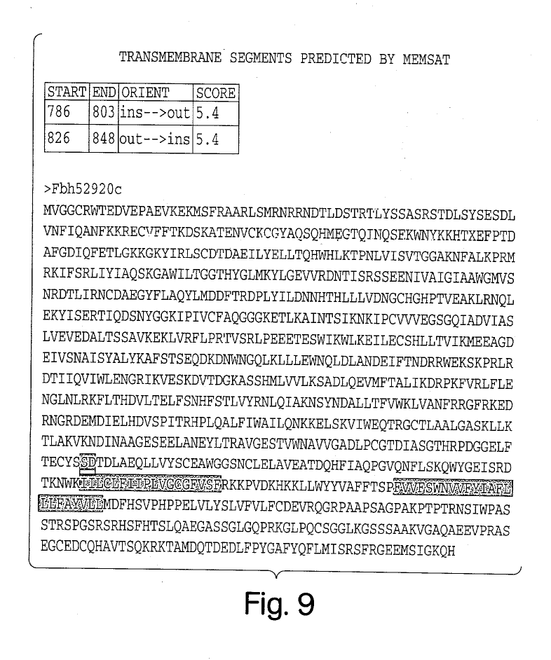

For example, the family of TLCC-4 and TLCC-5 polypeptides comprise at least one “transmembrane domain.” As used herein, the term “transmembrane domain” includes an amino acid sequence of about 20-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, alanines, valines, phenylalanines, prolines or methionines. Transmembrane domains are described in, for example, Zagotta W. N. et al, (1996) Annual Rev. Neurosci. 19: 235-263, the contents of which are incorporated herein by reference. Amino acid residues 440-461, 488-508, 520-540, 547-565, 590-609, and 652-676 of the human TLCC-4 polypeptide comprise transmembrane domains (see, e.g., FIGS. 4 and 6). Amino acid residues 786-803 and 826-848 of the human TLCC-5 polypeptide comprise transmembrane domains (see FIGS. 7 and 9). Accordingly, TLCC-4 and/or TLCC-5 polypeptides having at least 50-60% homology, preferably about 60-70%, more preferably about 70-80%, or about 80-90% homology with a transmembrane domain of human TLCC-4 and/or TLCC-5 are within the scope of the invention.

In another embodiment, a TLCC-4 molecule of the present invention is identified based on the presence of at least one “pore domain” between the fifth and sixth transmembrane domains. As used herein, the term “pore domain” includes an overall hydrophobic amino acid sequence which is located between two transmembrane domains of a calcium channel protein, preferably transmembrane domains 5 and 6, and which is believed to be a major determinant of ion selectivity and channel activity in calcium channels. Pore domains are described in, for example Vannier et al. (1998) J. Biol. Chem. 273: 8675-8679 and Phillips, A. M. et al. (1992) Neuron 8, 631-642, the contents of which are incorporated herein by reference. TLCC-4 molecules having at least one pore domain are within the scope of the invention. Amino acid residues 620-640 of the human TLCC-4 sequence (SEQ ID NO:2) comprise a pore domain (FIG. 4).

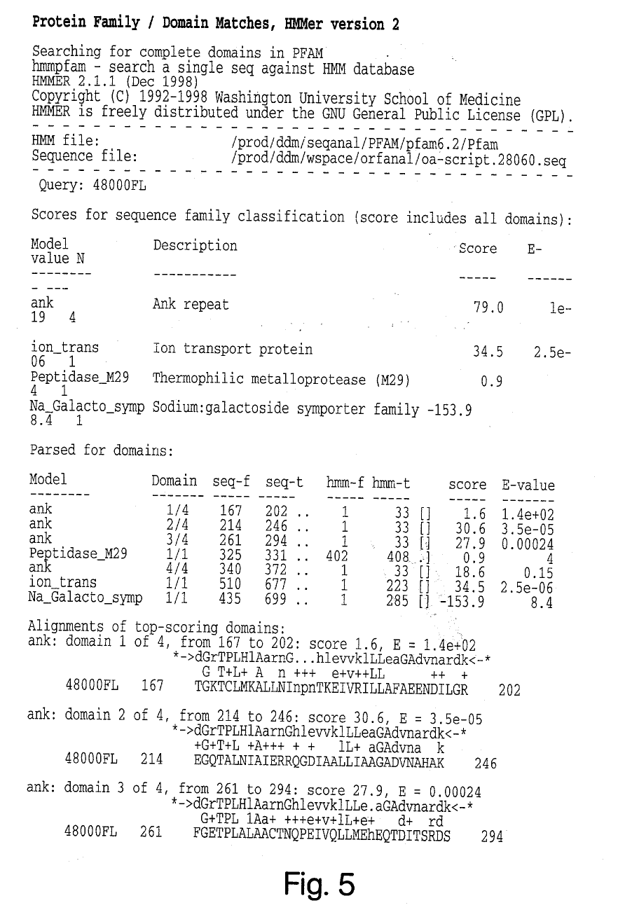

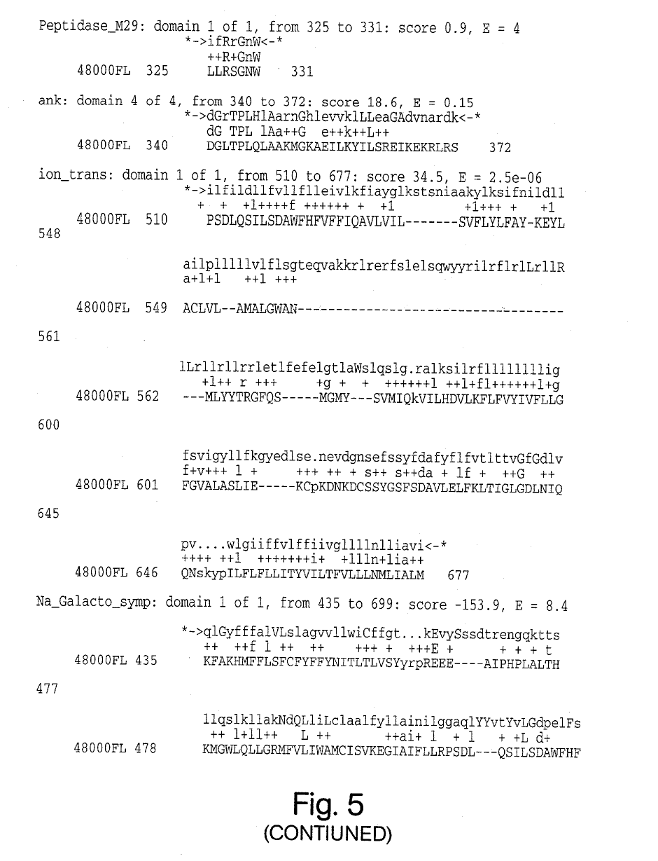

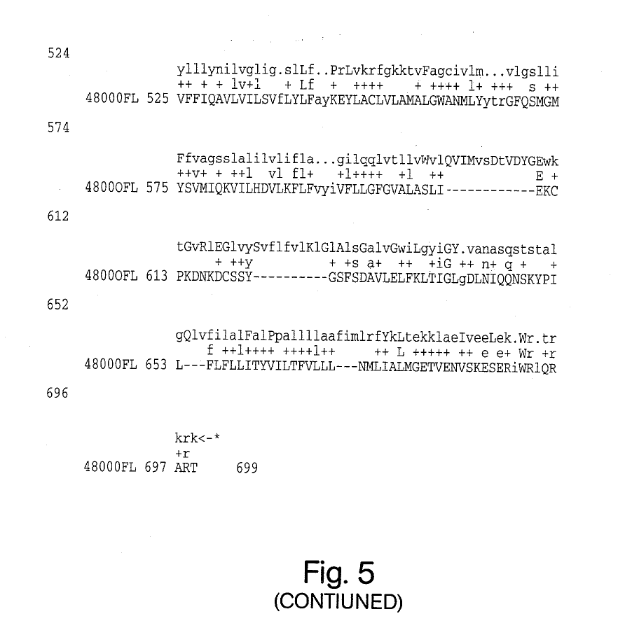

In another embodiment, a TLCC-4 molecule of the present invention is identified based on the presence of at least one “ankyrin repeat domain.” As used herein, the term “ankyrin repeat domain” includes an amino acid sequence of about 10-110 amino acid residues which serves as an ankyrin repeat. Preferably, an ankyrin repeat domain includes at least about 30 amino acid residues. To identify the presence of an ion transport domain in a TLCC-4 protein, and make the determination that a protein of interest has a particular profile, the amino acid sequence of the protein may be searched against a database of known protein domains (e.g., the HMM database). The ankyrin repeat domain (HMM) has been assigned the PFAM Accession PF00023 (at the PFAM website). A search was performed against the HMM database resulting in the identification of ankyrin repeat domains in the amino acid sequence of human TLCC-4 (SEQ ID NO:2) at about residues 167-202, 214-246, 261-294, and 340-372 of SEQ ID NO:2. The results of the search are set forth in FIG. 5.

In another embodiment, a TLCC-4 molecule of the present invention is identified based on the presence of at least one “ion transport protein domain.” As used herein, the term “ion transport protein domain” includes a protein domain having an amino acid sequence of about 100-200 amino acid residues which serves to transport ions. Preferably, an ion transport protein domain includes at least about 160 amino acid residues. To identify the presence of an ion transport protein domain in a TLCC-4 protein, and make the determination that a protein of interest has a particular profile, the amino acid sequence of the protein may be searched against a database of known protein domains (e.g., the HMM database). The ion transport domain (HMM) has been assigned the PFAM Accession PF00520 (at the world wide web address: genome.wustl.edu/Pfam/html). A search was performed against the HMM database resulting in the identification of an ion transport protein domain in the amino acid sequence of human TLCC-4 (SEQ ID NO:2) at about residues 510-677 of SEQ ID NO:2. The results of the search are set forth in FIG. 5.

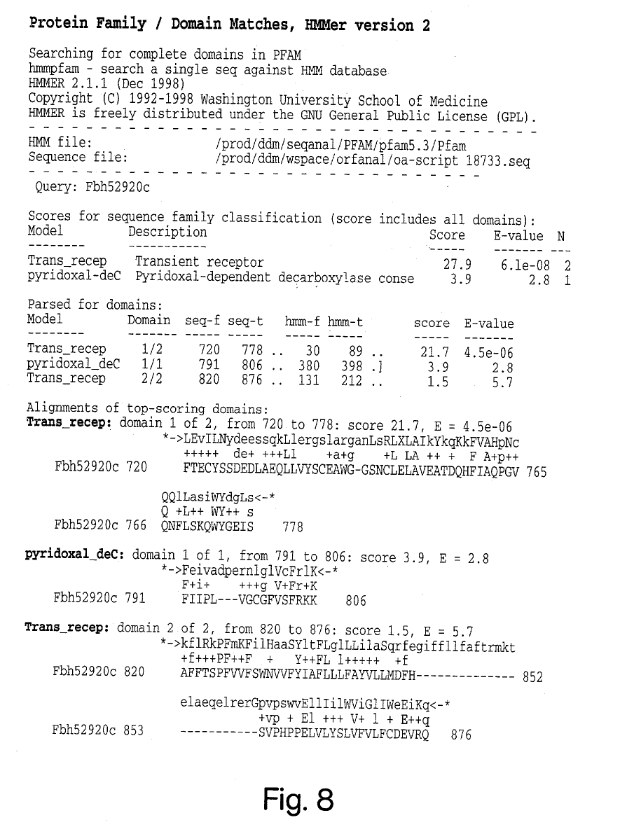

In another embodiment, a TLCC-5 molecule of the present invention is identified based on the presence of at least one “transient receptor domain.” As used herein, the term “transient receptor domain” includes a protein domain having an amino acid sequence of about 100-200 amino acid residues which is found in transient receptor potential (Trp) proteins and related ion channel proteins. Preferably, a transient receptor domain includes at least about 56-58 amino acid residues. To identify the presence of a transient receptor domain in a TLCC-5 protein, and make the determination that a protein of interest has a particular profile, the amino acid sequence of the protein may be searched against a database of known protein domains (e.g., the HMM database). The transient receptor domain (HMM) has been assigned the PFAM Accession PF02164 (at the world wide web address: genome.wustl.edu/Pfam/html). A search was performed against the HMM database resulting in the identification of transient receptor domains in the amino acid sequence of human TLCC-5 (SEQ ID NO:5) at about residues 720-778 and 820-876 of SEQ ID NO:5. The results of the search are set forth in FIG. 8.

A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al.(1994)J. Mol. Biol. 235:1501-1531; and Stultz et al.(1993) Protein Sci. 2:305-314, the contents of which are incorporated herein by reference. In a preferred embodiment, the TLCC-4 or TLCC-5 molecules of the invention include at least one transmembrane domain, at least one ankyrin repeat domain, at least one pore domain, at least one transient receptor domain, and/or at least one ion transport protein domain.

In a preferred embodiment, a TLCC-4 or TLCC-5 polypeptide includes at least one or more of the following domains: an ankyrin repeat domain, and/or a transmembrane domain, and/or a pore domain, and/or a transient receptor domain, and/or an ion transport protein domain, and has an amino acid sequence at least about 50%, 55%, 60%, 65%, 70%, 75%, 80%, 85%, 90%, 95%, 96%, 97%, 98%, 99% or more homologous or identical to the amino acid sequence of SEQ ID NO:2 or 5, or the amino acid sequences encoded by the DNA inserts of the plasmids deposited with ATCC as Accession Numbers . In yet another preferred embodiment, a TLCC-4 or TLCC-5 polypeptide includes at least one or more of the following domains: an ankyrin repeat domain, and/or a transmembrane domain, and/or a pore domain, and/or a transient receptor domain, and/or an ion transport protein domain, and is encoded by a nucleic acid molecule having a nucleotide sequence which hybridizes under stringent hybridization conditions to a complement of a nucleic acid molecule comprising the nucleotide sequence of SEQ ID NO:1, 3, 4, or 6. In another preferred embodiment, a TLCC-4 or TLCC-5 polypeptide includes at least one or more of the following domains: an ankyrin repeat domain, and/or a transmembrane domain, and/or a pore domain, and/or a transient receptor domain, and/or an ion transport protein domain, and has a TLCC-4 or TLCC-5 activity.

As used interchangeably herein, a “TLCC-4 or TLCC-5 activity”, “biological activity of TLCC-4 or TLCC-5 ” or “functional activity of TLCC-4 or TLCC-5”, refers to an activity exerted by a TLCC-4 or TLCC-5 polypeptide or nucleic acid molecule on a TLCC-4 or TLCC-5 responsive cell or tissue, or on a TLCC-4 or TLCC-5 polypeptide substrate, as determined in vivo, or in vitro, according to standard techniques. In one embodiment, a TLCC-4 or TLCC-5 activity is a direct activity, such as an association with a TLCC-4-target molecule or TLCC-5-target molecule. As used herein, a “target molecule” or “binding partner” is a molecule with which a TLCC-4 or TLCC-5 polypeptide binds or interacts in nature, such that TLCC-4-mediated or TLCC-5-mediated function is achieved. A TLCC-4 or TLCC-5 target molecule can be a non-TLCC-4 or non-TLCC-5 molecule or a TLCC-4 or TLCC-5 polypeptide or polypeptide of the present invention. In an exemplary embodiment, a TLCC-4 or TLCC-5 target molecule is a TLCC-4 or TLCC-5 ligand, e.g., a calcium channel ligand such as calcium. Alternatively, a TLCC-4 or TLCC-5 activity is an indirect activity, such as a cellular signaling activity mediated by interaction of the TLCC-4 or TLCC-5 polypeptide with a TLCC-4 or TLCC-5 ligand. The biological activities of TLCC-4 or TLCC-5 are described herein. For example, the TLCC-4 or TLCC-5 polypeptides of the present invention can have one or more of the following activities: (1) modulate membrane excitability, (2) influence the resting potential of membranes, (3) modulate wave forms and frequencies of action potentials, (4) modulate thresholds of excitation, (5) modulate neurite outgrowth and synaptogenesis, (6) modulate signal transduction, and (7) participate in nociception.

Accordingly, another embodiment of the invention features isolated TLCC-4 or TLCC-5 polypeptides and polypeptides having a TLCC-4 or TLCC-5 activity. Preferred polypeptides are TLCC-4 or TLCC-5 polypeptides having at least one or more of the following domains: an ankyrin repeat domain, and/or a transmembrane domain, and/or a pore domain, and/or a transient receptor domain, and/or an ion transport protein domain and, preferably, a TLCC-4 or TLCC-5 activity.

Additional preferred polypeptides have one or more of the following domains: an ankyrin repeat domain, a transmembrane domain, a pore domain, a transient receptor domain, and/or an ion transport protein domain, and are, preferably, encoded by a nucleic acid molecule having a nucleotide sequence which hybridizes under stringent hybridization conditions to a nucleic acid molecule comprising the nucleotide sequence of SEQ ID NO:1, 3, 4, or 6.

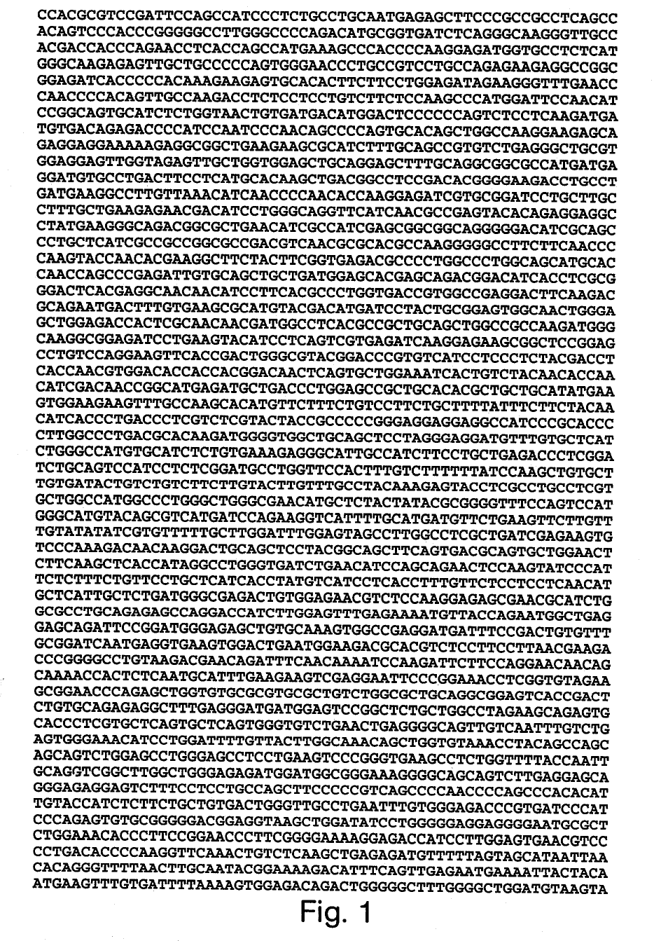

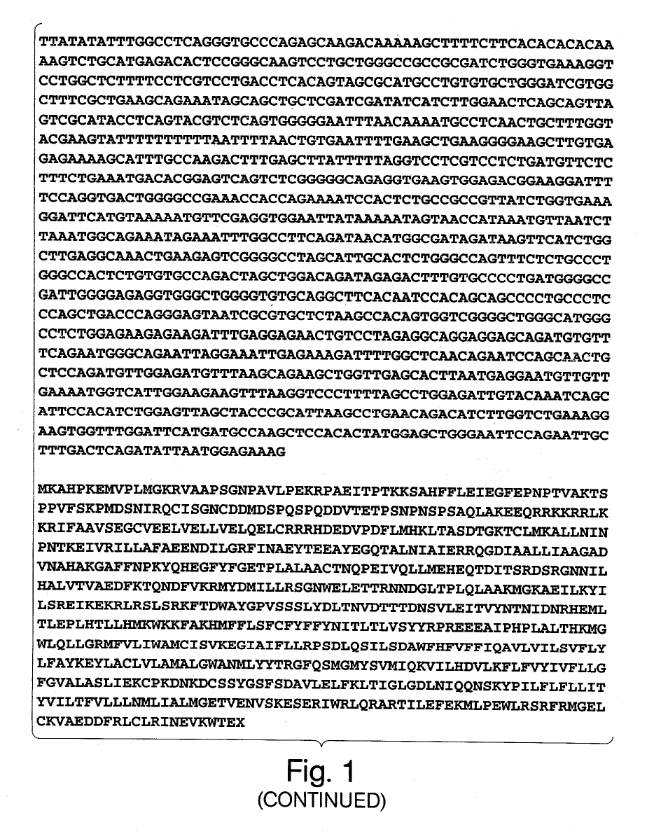

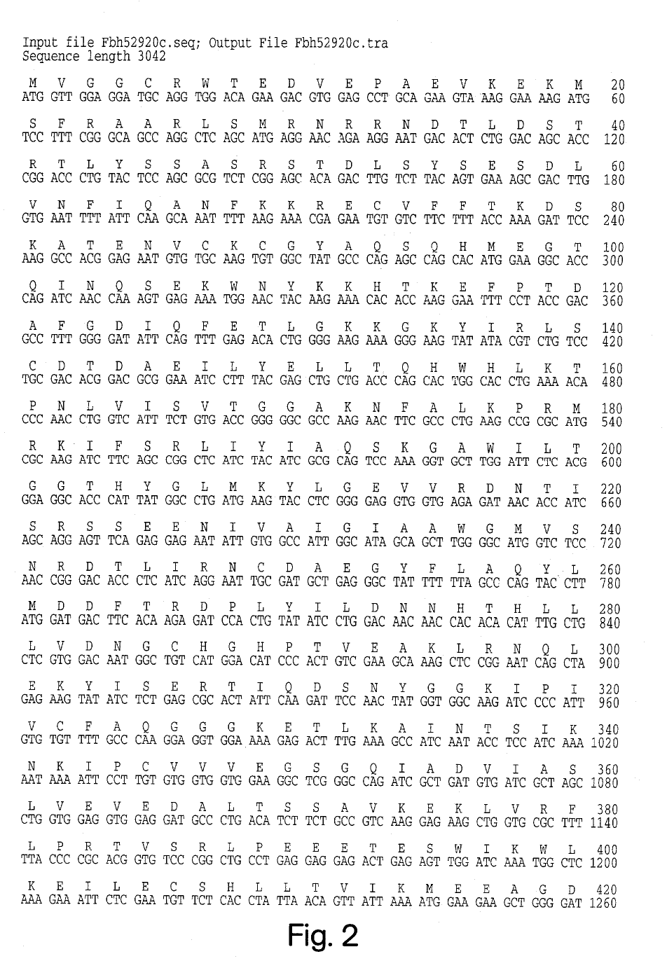

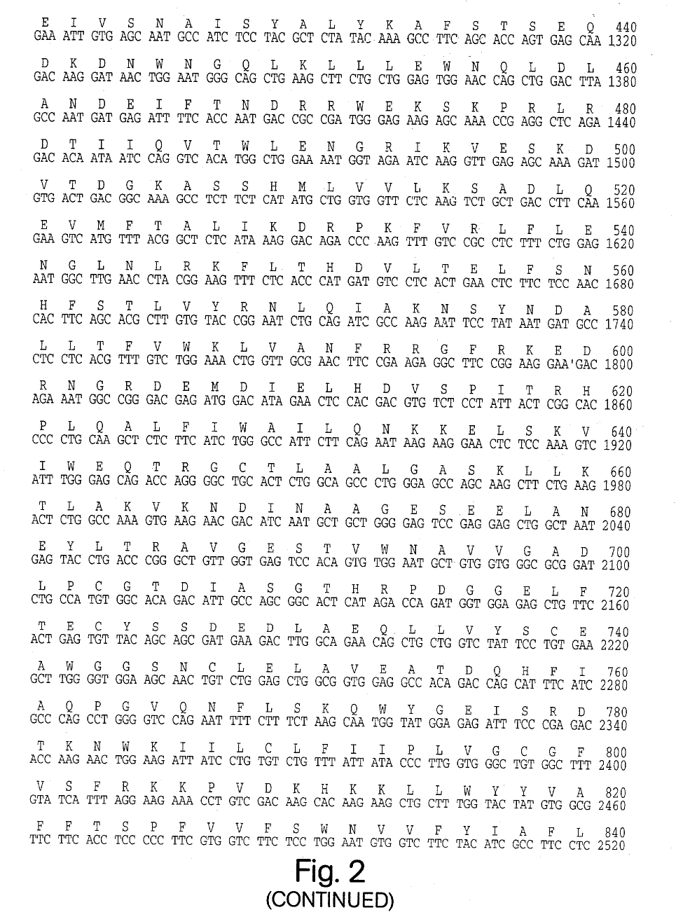

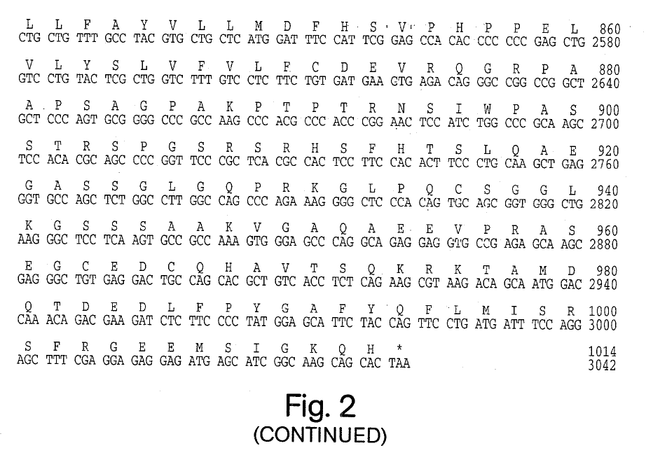

The nucleotide sequence of the isolated human TLCC-4 and TLCC-5 cDNA and the predicted amino acid sequences of the human TLCC-4 and TLCC-5 polypeptides are shown in FIGS. 1 and 2, and in SEQ ID NOs: 1 and 2, and 3 and 4, respectively. Plasmids containing the nucleotide sequences encoding human TLCC-4 and TLCC-5 were deposited with the American Type Culture Collection (ATCC), 10801 University Boulevard, Manassas, Va. 20110-2209, on______ and assigned Accession Numbers______ . These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

The human TLCC-4 gene, which is approximately 3321 nucleotides in length, encodes a polypeptide which is approximately 751 amino acid residues in length. The human TLCC-5 gene, which is approximately 3042 nucleotides in length, encodes a polypeptide which is approximately 1013 amino acid residues in length.

Various aspects of the invention are described in further detail in the following subsections:

I. Isolated Nucleic Acid Molecules

One aspect of the invention pertains to isolated nucleic acid molecules that encode TLCC-4 or TLCC-5 polypeptides or biologically active portions thereof, as well as nucleic acid fragments sufficient for use as hybridization probes to identify TLCC-4-encoding or TLCC-5-encoding nucleic acid molecules (e.g., TLCC-4 or TLCC-5 mRNA) and fragments for use as PCR primers for the amplification or mutation of TLCC-4 or TLCC-5 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TLCC-4 or TLCC-5 nucleic acid molecule can contain less than about 5 kb, 4 kb, 3 kb, 2 kb, 1 k b, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 3, 4, or 6, or the nucleotide sequences of the DNA inserts of the plasmids deposited with ATCC as Accession Numbers______, or a portion thereof, can be isolated using standard molecular biology techniques and the sequence information provided herein. Using all or a portion of the nucleic acid sequence of SEQ ID NO:1, 3, 4, or 6, or the nucleotide sequences of the DNA inserts of the plasmids deposited with ATCC as Accession Numbers______, as a hybridization probe, TLCC-4 or TLCC-5 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 4, or 6, or the nucleotide sequences of the DNA inserts of the plasmids deposited with ATCC as Accession Numbers______ can be isolated by the polymerase chain reaction (PCR) using synthetic oligonucleotide primers designed based upon the sequence of SEQ ID NO:1, 3, 4, or 6, or the nucleotide sequences of the DNA inserts of the plasmids deposited with ATCC as Accession Numbers______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to TLCC-4 or TLCC-5 nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:1 or SEQ ID NO:4. The sequence of SEQ ID NO:1 corresponds to the human TLCC-4. This cDNA comprises sequences encoding the human TLCC-4 polypeptide (i.e., “the coding region”, from nucleotides 146-2368) as well as 5′ untranslated sequences (nucleotides 1-145) and 3′ untranslated sequences (nucleotides 2369-4586). Alternatively, the nucleic acid molecule can comprise only the coding region of SEQ ID NO:1 (e.g., nucleotides 146-2368, corresponding to SEQ ID NO:3). Accordingly, in another embodiment, the isolated nucleic acid molecule comprises SEQ ID NO:3 and nucleotides 1-145 and 2369-4586 of SEQ ID NO:1. The sequence of SEQ ID NO:4 corresponds to the human TLCC-5. This cDNA comprises sequences encoding the human TLCC-5 polypeptide (i.e., “the coding region”, from nucleotides 1-3042). In yet another embodiment, the nucleic acid molecule consists of the nucleotide sequence set forth as SEQ ID NO:1, 3, 4, or 6.

In still another embodiment, an isolated nucleic acid molecule of the invention comprises a nucleic acid molecule which is a complement of the nucleotide sequence shown in SEQ ID NO:1, 3, 4, or 6, or the nucleotide sequences of the DNA inserts of the plasmids deposited with ATCC as Accession Numbers______, or a portion of any of these nucleotide sequences. A nucleic acid molecule which is complementary to the nucleotide sequence shown in SEQ ID NO:1, 3, 4, or 6, or the nucleotide sequences of the DNA inserts of the plasmids deposited with ATCC as Accession Numbers______, is one which is sufficiently complementary to the nucleotide sequence shown in SEQ ID NO:1, 3, 4, or 6, or the nucleotide sequences of the DNA inserts of the plasmids deposited with ATCC as Accession Numbers______, such that it can hybridize to the nucleotide sequence shown in SEQ ID NO:1, 3, 4, or 6, or the nucleotide sequences of the DNA inserts of the plasmids deposited with ATCC as Accession Numbers______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99% or more identical to th entire length of the nucleotide sequence shown in SEQ ID NO:1, 3, 4, or 6, (e.g., the entire length of the nucleotide sequence), or to the nucleotide sequences (e.g., the entire length of the nucleotide sequence) of the DNA inserts of the plasmids deposited with ATCC as Accession Numbers______, or to a portion or complement of any of these nucleotide sequences. In one embodiment, a nucleic acid molecule of the present invention comprises a nucleotide sequence which is at least 50, 100, 150, 200, 250, 300, 350, 400, 450, 500, 550, 600, 650, 700, 750, 800, 850, 900, 950, 1000, 1050, 1100, 1150, 1200, 1250, 1300, 11350, 1400, 1450, 1500 or more nucleotides in length and hybridizes under stringent hybridization conditions to a complement of a nucleic acid molecule of SEQ ID NO:1, 3, 4, or 6 or the nucleotide sequences of the DNA inserts of the plasmids deposited with ATCC as Accession Numbers______.

Moreover, the nucleic acid molecule of the invention can comprise only a portion of the nucleic acid sequence of SEQ ID NO:1, 3, 4, or 6, or the nucleotide sequences of the DNA inserts of the plasmids deposited with ATCC as Accession Numbers______, for example, a fragment which can be used as a probe or primer or a fragment encoding a portion of a TLCC-4 or TLCC-5 polypeptide, e.g., a biologically active portion of a TLCC-4 or TLCC-5 polypeptide. The nucleotide sequence determined from the cloning of the TLCC-4 or TLCC-5 gene allows for the generation of probes and primers designed for use in identifying and/or cloning other TLCC-4 or TLCC-5 family members, as well as TLCC-4 or TLCC-5 homologues from other species. The probe/primer typically comprises substantially purified oligonucleotide. The probe/primer (e.g., oligonucleotide) typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, or 100 or more consecutive nucleotides of a sense sequence of SEQ ID NO:1, 3, 4, or 6, or the nucleotide sequences of the DNA inserts of the plasmids deposited with ATCC as Accession Numbers______, of an anti-sense sequence of SEQ ID NO:1, 3, 4, or 6, or the nucleotide sequences of the DNA inserts of the plasmids deposited with ATCC as Accession Numbers______, or of a naturally occurring allelic variant or mutant of SEQ ID NO:1, 3, 4, or 6, or the nucleotide sequences of the DNA inserts of the plasmids deposited with ATCC as Accession Numbers______.

Exemplary probes or primers are at least (or no greater than)12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the TLCC-4 or TLCC-5 nucleotide sequences can be used to detect (e.g., specifically detect) transcripts or genomic sequences encoding the same or homologous polypeptides. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in PCR, which can be used to amplify a selected region of a TLCC-4 or TLCC-5 sequence, e.g., a domain, region, site or other sequence described herein. The primers should be at least 5, 10, or 50 base pairs in length and less than 100, or less than 200, base pairs in length. The primers should be identical, or differ by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which misexpress a TLCC-4 or TLCC-5 polypeptide, such as by measuring a level of a TLCC-4-encoding or TLCC-5-encoding nucleic acid in a sample of cells from a subject e.g., detecting TLCC-4 or TLCC-5 mRNA levels or determining whether a genomic TLCC-4 or TLCC-5 gene has been mutated or deleted.

A nucleic acid fragment encoding a “biologically active portion of a TLCC-4 or TLCC-5 polypeptide” can be prepared by isolating a portion of the nucleotide sequence of SEQ ID NO:1, 3, 4, or 6, or the nucleotide sequences of the DNA inserts of the plasmids deposited with ATCC as Accession Numbers______, which encodes a polypeptide having a TLCC-4 or TLCC-5 biological activity (the biological activities of the TLCC-4 or TLCC-5 polypeptides are described herein), expressing the encoded portion of the TLCC-4 or TLCC-5 polypeptide (e.g., by recombinant expression in vitro) and assessing the activity of the encoded portion of the TLCC-4 or TLCC-5 polypeptide. In an exemplary embodiment, the nucleic acid molecule is at least 50-100, 100-250, 250-500, 500-700, 700-1000, 1000-1250, 1250-1500, 1500-1750, 1750-2000, 2000-2250, 2250-2500, 2500-2750, 2750-300 or more nucleotides in length and encodes a protein having a TLCC-4 or TLCC-5 activity (as described herein).

The invention further encompasses nucleic acid molecules that differ from the nucleotide sequence shown in SEQ ID NO:1, 3, 4, or 6, or the nucleotide sequences of the DNA inserts of the plasmids deposited with ATCC as Accession Numbers______. Such differences can be due to degeneracy of the genetic code, thus resulting in a nucleic acid which encodes the same TLCC-4 or TLCC-5 polypeptides as those encoded by the nucleotide sequence shown in SEQ ID NO:1, 3, 4, or 6, or the nucleotide sequences of the DNA inserts of the plasmids deposited with ATCC as Accession Numbers______. In another embodiment, an isolated nucleic acid molecule of the invention has a nucleotide sequence encoding a polypeptide having an amino acid sequence which differs by at least 1, but no greater than 5, 10, 20, 50 or 100 amino acid residues from the amino acid sequence shown in SEQ ID NO:2 or 5, or the amino acid sequences encoded by the DNA inserts of the plasmids deposited with the ATCC as Accession Numbers______. In yet another embodiment, the nucleic acid molecule encodes the amino acid sequence of human TLCC-4 or human TLCC-5. If an alignment is needed for this comparison, the sequences should be aligned for maximum homology.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared to the encoded product).

Allelic variants result, for example, from DNA sequence polymorphisms within a population (e.g., the human population) that lead to changes in the amino acid sequences of the TLCC-4 or TLCC-5 polypeptides. Such genetic polymorphisms in the TLCC-4 or TLCC-5 genes may exist among individuals within a population due to natural allelic variation. As used herein, the terms “gene” and “recombinant gene” refer to nucleic acid molecules which include an open reading frame encoding a TLCC-4 or TLCC-5 polypeptide, preferably a mammalian TLCC-4 or TLCC-5 polypeptide, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:2 or 5, or amino acid sequences encoded by the DNA inserts of the plasmids deposited with ATCC as Accession Numbers______, wherein the nucleic acid molecule hybridizes to a complement of a nucleic acid molecule comprising SEQ ID NO:1, 3, 4, or 6, for example, under stringent hybridization conditions.

Allelic variants of human TLCC-4 or TLCC-5 include both functional and non-functional TLCC-4 or TLCC-5 polypeptides. Functional allelic variants are naturally occurring amino acid sequence variants of the human TLCC-4 or TLCC-5 polypeptide that maintain the ability to bind a TLCC-4 or TLCC-5 ligand or substrate and/or modulate membrane excitability or signal transduction. Functional allelic variants will typically contain only conservative substitution of one or more amino acids of SEQ ID NO:2 or 5, or substitution, deletion or insertion of non-critical residues in non-critical regions of the polypeptide.

Non-functional allelic variants are naturally occurring amino acid sequence variants of the human TLCC-4 or TLCC-5 polypeptide that do not have the ability to form functional calcium channels or to modulate membrane excitability. Non-functional allelic variants will typically contain a non-conservative substitution, a deletion, or insertion or premature truncation of the amino acid sequence of SEQ ID NO:2 or 5, or a substitution, insertion or deletion in critical residues or critical regions.

The present invention further provides non-human orthologues (e.g., non-human orthologues of the human TLCC-4 or TLCC-5 polypeptides). Orthologues of the human TLCC-4 or TLCC-5 polypeptide are polypeptides that are isolated from non-human organisms and possess the same TLCC-4 or TLCC-5 ligand binding and/or modulation of membrane excitation mechanisms of the human TLCC-4 or TLCC-5 polypeptide. Orthologues of the human TLCC-4 or TLCC-5 polypeptide can readily be identified as comprising an amino acid sequence that is substantially identical to SEQ ID NO:2 or 5.

Moreover, nucleic acid molecules encoding other TLCC-4 or TLCC-5 family members and, thus, which have a nucleotide sequence which differs from the TLCC-4 or TLCC-5 sequences of SEQ ID NO:1, 3, 4, or 6, or the nucleotide sequences of the DNA inserts of the plasmids deposited with ATCC as Accession Numbers_______ are intended to be within the scope of the invention. For example, another TLCC-4 or TLCC-5 cDNA can be identified based on the nucleotide sequence of human TLCC-4 or TLCC-5. Moreover, nucleic acid molecules encoding TLCC-4 or TLCC-5 polypeptides from different species, and which, thus, have a nucleotide sequence which differs from the TLCC-4 or TLCC-5 sequences of SEQ ID NO:1, 3, 4, or 6, or the nucleotide sequences of the DNA inserts of the plasmids deposited with ATCC as Accession Numbers______ are intended to be within the scope of the invention. For example, a mouse TLCC-4 or TLCC-5 cDNA can be identified based on the nucleotide sequence of a human TLCC-4 or TLCC-5.

Nucleic acid molecules corresponding to natural allelic variants and homologues of the TLCC-4 or TLCC-5 cDNAs of the invention can be isolated based on their homology to the TLCC-4 or TLCC-5 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the TLCC-4 or TLCC-5 cDNAs of the invention can further be isolated by mapping to the same chromosome or locus as the TLCC-4 or TLCC-5 gene.

Orthologues, homologues and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, 3, 4, or 6, or the nucleotide sequences of the DNA inserts of the plasmids deposited with ATCC as Accession Numbers______ . In another embodiment, the nucleic acid is at least 100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1070, 1070-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 4000-4050, 4050-4100, 4100-4150, 4150-4200, 4200-4250, 4250-4300, 4300-4350, 4350-4400, 4400-4450, 4450-4500 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(°C.)=2(# of A+T bases)+4(# of G+C bases). For hybrids between 18 and 49 base pairs in length, Tₘ(°C.) =81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see, e.g., Church and Gilbert (1984) Proc. Natl. Acad. Sci. USA 81:1991-1995, (or alternatively 0.2×SSC, 1% SDS).

Preferably, an isolated nucleic acid molecule of the invention that hybridizes under stringent conditions to the sequence of SEQ ID NO:1, 3, 4, or 6 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural polypeptide).

In addition to naturally-occurring allelic variants of the TLCC-4 or TLCC-5 sequences that may exist in the population, the skilled artisan will further appreciate that changes can be introduced by mutation into the nucleotide sequences of SEQ ID NO:1, 3, 4, or 6, or the nucleotide sequences of the DNA inserts of the plasmids deposited with ATCC as Accession Numbers______, thereby leading to changes in the amino acid sequence of the encoded TLCC-4 or TLCC-5 polypeptides, without altering the functional ability of the TLCC-4 or TLCC-5 polypeptides. For example, nucleotide substitutions leading to amino acid substitutions at “non-essential” amino acid residues can be made in the sequence of SEQ ID NO:1, 3, 4, or 6, or the nucleotide sequences of the DNA inserts of the plasmids deposited with ATCC as Accession Numbers______. A “non-essential” amino acid residue is a residue that can be altered from the wild-type sequence of TLCC-4 or TLCC-5 (e.g., the sequence of SEQ ID NO:2 or 5) without altering the biological activity, whereas an “essential” amino acid residue is required for biological activity. For example, amino acid residues that are conserved among the TLCC-4 or TLCC-5 polypeptides of the present invention, e.g., those present in a transmembrane domain, are predicted to be particularly unamenable to alteration. Furthermore, additional amino acid residues that are conserved between the TLCC-4 or TLCC-5 polypeptides of the present invention and other members of the TLCC-4 or TLCC-5 family are not likely to be amenable to alteration.

Accordingly, another aspect of the invention pertains to nucleic acid molecules encoding TLCC-4 or TLCC-5 polypeptides that contain changes in amino acid residues that are not essential for activity. Such TLCC-4 or TLCC-5 polypeptides differ in amino acid sequence from SEQ ID NO:2 or 5, yet retain biological activity. In one embodiment, the isolated nucleic acid molecule comprises a nucleotide sequence encoding a polypeptide, wherein the polypeptide comprises an amino acid sequence at least about 50%, 55%, 60%, 65%, 70%, 75%, 80%, 85%, 90%, 95%, 96%, 97%, 98%, 99% or more identical to SE NO:2 or 5 (e.g., to the entire length of SEQ ID NO:2 or 5).

An isolated nucleic acid molecule encoding a TLCC-4 or TLCC-5 polypeptide identical to the polypeptide of SEQ ID NO:2 or 5, can be created by introducing one or more nucleotide substitutions, additions or deletions into the nucleotide sequence of SEQ ID NO:1, 3, 4, or 6, or the nucleotide sequences of the DNA inserts of the plasmids deposited with ATCC as Accession Numbers______, such that one or more amino acid substitutions, additions or deletions are introduced into the encoded polypeptide. Mutations can be introduced into SEQ ID NO:1, 3, 4, or 6, or the nucleotide sequences of the DNA inserts of the plasmids deposited with ATCC as Accession Numbers______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a TLCC-4 or TLCC-5 polypeptide is preferably replaced with another amino acid residue from the same side chain family. Alternatively, in another embodiment, mutations can be introduced randomly along all or part of a TLCC-4 or TLCC-5 coding sequence, such as by saturation mutagenesis, and the resultant mutants can be screened for TLCC-4 or TLCC-5 biological activity to identify mutants that retain activity. Following mutagenesis of SEQ ID NO:1, 3, 4, or 6, or the nucleotide sequences of the DNA inserts of the plasmids deposited with ATCC as Accession Numbers______, the encoded polypeptide can be expressed recombinantly and the activity of the polypeptide can be determined.

In a preferred embodiment, a mutant TLCC-4 or TLCC-5 polypeptide can be assayed for the ability to (1) modulate membrane excitability, (2) influence the resting potential of membranes, (3) modulate wave forms and frequencies of action potentials, (4) modulate thresholds of excitation, (5) modulate neurite outgrowth and synaptogenesis, (6) modulate signal transduction, and (7) participate in nociception.

In addition to the nucleic acid molecules encoding TLCC-4 or TLCC-5 polypeptides described above, another aspect of the invention pertains to isolated nucleic acid molecules which are antisense thereto. In an exemplary embodiment, the invention provides an isolated nucleic acid molecule which is antisense to a TLCC-4 or TLCC-5 nucleic acid molecule (e.g., is antisense to the coding strand of a TLCC-4 or TLCC-5 nucleic acid molecule). An “antisense” nucleic acid comprises a nucleotide sequence which is complementary to a “sense” nucleic acid encoding a polypeptide, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TLCC-4 or TLCC-5 coding strand, or to only a portion thereof. In one embodiment, an antisense nucleic acid molecule is antisense to a “coding region” of the coding strand of a nucleotide sequence encoding TLCC-4 or TLCC-5. The term “coding region” refers to the region of the nucleotide sequence comprising codons which are translated into amino acid residues (e.g., the coding region of human TLCC-4 or TLCC-5 corresponds to SEQ ID NO:3 or SEQ ID NO:6, respectively). In another embodiment, the antisense nucleic acid molecule is antisense to a “noncoding region” of the coding strand of a nucleotide sequence encoding TLCC-4 or TLCC-5. The term “noncoding region” refers to 5′ and 3′ sequences which flank the coding region that are not translated into amino acids (i.e., also referred to as 5′ and 3′ untranslated regions).

Given the coding strand sequences encoding TLCC-4 or TLCC-5 disclosed herein (e.g., SEQ ID NO:3 or 6), antisense nucleic acids of the invention can be designed according to the rules of Watson and Crick base pairing. The antisense nucleic acid molecule can be complementary to the entire coding region of TLCC-4 or TLCC-5 mRNA, but more preferably is an oligonucleotide which is antisense to only a portion of the coding or noncoding region of TLCC-4 or TLCC-5 mRNA. For example, the antisense oligonucleotide can be complementary to the region surrounding the translation start site of TLCC-4 or TLCC-5 mRNA (e.g., between the −10 and +10 regions of the start site of a gene nucleotide sequence). An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TLCC-4 or TLCC-5 polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave TLCC-4 or TLCC-5 mRNA transcripts to thereby inhibit translation of TLCC-4 or TLCC-5 mRNA. A ribozyme having specificity for a TLCC-4-encoding or TLCC-5-encoding nucleic acid can be designed based upon the nucleotide sequence of a TLCC-4 or TLCC-5 cDNA disclosed herein (i.e., SEQ ID NO:1, 3, 4, or 6, or the nucleotide sequences of the DNA inserts of the plasmids deposited with ATCC as Accession Numbers______). For example, a derivative of a Tetrahymena L-19 IVS RNA can be constructed in which the nucleotide sequence of the active site is complementary to the nucleotide sequence to be cleaved in a TLCC-4-encoding or TLCC-5-encoding mRNA (see, e.g., Cech et al. U.S. Pat. No. 4,987,071; and Cech et al. U.S. Pat. No. 5,116,742). Alternatively, TLCC-4 or TLCC-5 mRNA can be used to select a catalytic RNA having a specific ribonuclease activity from a pool of RNA molecules. See, e.g., Bartel, D. and Szostak, J. W. (1993) Science 261:1411-1418.

Alternatively, TLCC-4 or TLCC-5 gene expression can be inhibited by targeting nucleotide sequences complementary to the regulatory region of the TLCC-4 or TLCC-5 (e.g., the TLCC-4 or TLCC-5 promoter and/or enhancers) to form triple helical structures that prevent transcription of the TLCC-4 or TLCC-5 gene in target cells (see, generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

In yet another embodiment, the TLCC-4 or TLCC-5 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see, e.g., Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of TLCC-4 or TLCC-5 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TLCC-4 or TLCC-5 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of TLCC-4 or TLCC-5 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of TLCC-4 or TLCC-5 nucleic acid molecules can be generated which may combine the advantageous properties of PNA and DNA. Such chimeras allow DNA recognition enzymes, (e.g., RNase H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (see, e.g., Krol et al. (1988) Bio-Techniques 6:958-976) or intercalating agents (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous TLCC-4 or TLCC-5 gene within a cell line or microorganism may be modified by inserting a heterologous DNA regulatory element into the genome of a stable cell line or cloned microorganism such that the inserted regulatory element is operatively linked with the endogenous TLCC-4 or TLCC-5 gene. For example, an endogenous TLCC-4 or TLCC-5 gene which is normally “transcriptionally silent”, i.e., a TLCC-4 or TLCC-5 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous TLCC-4 or TLCC-5 gene may be activated by insertion of a promiscuous regulatory element that works across cell types.

A heterologous regulatory element may be inserted into a stable cell line or cloned microorganism, such that it is operatively linked with an endogenous TLCC-4 or TLCC-5 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated TLCC-4 and TLCC-5 Polypeptides and Anti-TLCC-4 and Anti-TLCC-5 Antibodies

One aspect of the invention pertains to isolated or recombinant TLCC-4 and TLCC-5 polypeptides, and biologically active portions thereof, as well as polypeptide fragments suitable for use as immunogens to raise anti-TLCC-4 or anti-TLCC-5 antibodies. In one embodiment, native TLCC-4 or TLCC-5 polypeptides can be isolated from cells or tissue sources by an appropriate purification scheme using standard polypeptide purification techniques. In another embodiment, TLCC-4 or TLCC-5 polypeptides are produced by recombinant DNA techniques. Alternative to recombinant expression, a TLCC-4 or TLCC-5 polypeptide can be synthesized chemically using standard peptide synthesis techniques.

An “isolated” or “purified” polypeptide or biologically active portion thereof is substantially free of cellular material or other contaminating proteins from the cell or tissue source from which the TLCC-4 or TLCC-5 polypeptide is derived, or substantially free from chemical precursors or other chemicals when chemically synthesized. The language “substantially free of cellular material” includes preparations of TLCC-4 or TLCC-5 polypeptide in which the polypeptide is separated from cellular components of the cells from which it is isolated or recombinantly produced. In one embodiment, the language “substantially free of cellular material” includes preparations of TLCC-4 or TLCC-5 polypeptide having less than about 30% (by dry weight) of non-TLCC-4 or non-TLCC-5 polypeptide (also referred to herein as a “contaminating protein”), more preferably less than about 20% of non-TLCC-4 or non-TLCC-5 polypeptide, still more preferably less than about 10% of non-TLCC-4 or non-TLCC-5 polypeptide, and most preferably less than about 5% non-TLCC-4 or non-TLCC-5 polypeptide. When the TLCC-4 or TLCC-5 polypeptide or biologically active portion thereof is recombinantly produced, it is also preferably substantially free of culture medium, i.e., culture medium represents less than about 20%, more preferably less than about 10%, and most preferably less than about 5% of the volume of the polypeptide preparation.

The language “substantially free of chemical precursors or other chemicals” includes preparations of TLCC-4 or TLCC-5 polypeptide in which the polypeptide is separated from chemical precursors or other chemicals which are involved in the synthesis of the polypeptide. In one embodiment, the language “substantially free of chemical precursors or other chemicals” includes preparations of TLCC-4 or TLCC-5 polypeptide having less than about 30% (by dry weight) of chemical precursors or non-TLCC-4 or non-TLCC-5 chemicals, more preferably less than about 20% chemical precursors or non-TLCC-4 or non-TLCC-5 chemicals, still more preferably less than about 10% chemical precursors or non-TLCC-4 or non-TLCC-5 chemicals, and most preferably less than about 5% chemical precursors or non-TLCC-4 or non-TLCC-5 chemicals.

As used herein, a “biologically active portion” of a TLCC-4 or TLCC-5 polypeptide includes a fragment of a TLCC-4 or TLCC-5 polypeptide which participates in an interaction between a TLCC-4 or TLCC-5 molecule and a non-TLCC-4 or non-TLCC-5 molecule. Biologically active portions of a TLCC-4 or TLCC-5 polypeptide include peptides comprising amino acid sequences sufficiently identical to or derived from the amino acid sequence of the TLCC-4 or TLCC-5 polypeptide, e.g., the amino acid sequence shown in SEQ ID NO:2 or 5, which include less amino acids than the full length TLCC-4 or TLCC-5 polypeptides, and exhibit at least one activity of a TLCC-4 or TLCC-5 polypeptide. Typically, biologically active portions comprise a domain or motif with at least one activity of the TLCC-4 or TLCC-5 polypeptide, e.g., modulating membrane excitation mechanisms. A biologically active portion of a TLCC-4 or TLCC-5 polypeptide can be a polypeptide which is, for example, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 or more amino acids in length. Biologically active portions of a TLCC-4 or TLCC-5 polypeptide can be used as targets for developing agents which modulate a TLCC-4 or TLCC-5 mediated activity, e.g., a membrane excitation mechanism.

In one embodiment, a biologically active portion of a TLCC-4 or TLCC-5 polypeptide comprises at least one transmembrane domain. It is to be understood that a preferred biologically active portion of a TLCC-4 or TLCC-5 polypeptide of the present invention comprises at least one or more of the following domains: an ankyrin repeat domain, a transmembrane domain, a pore domain, a transient receptor domain, and/or an ion transport protein domain. Moreover, other biologically active portions, in which other regions of the polypeptide are deleted, can be prepared by recombinant techniques and evaluated for one or more of the functional activities of a native TLCC-4 or TLCC-5 polypeptide.

Another aspect of the invention features fragments of the protein having the amino acid sequence of SEQ ID NO:2 or 5, for example, for use as immunogens. In one embodiment, a fragment comprises at least 5 amino acids (e.g., contiguous or consecutive amino acids) of the amino acid sequence of SEQ ID NO:2 or 5, or amino acid sequences encoded by the DNA inserts of the plasmids deposited with the ATCC as Accession Numbers______. In another embodiment, a fragment comprises at least 10, 15, 20, 25, 30, 35, 40, 45, 50 or more amino acids (e.g., contiguous or consecutive amino acids) of the amino acid sequence of SEQ ID NO:2 or 5, or amino acid sequences encoded by the DNA inserts of the plasmids deposited with the ATCC as Accession Numbers______.

In a preferred embodiment, a TLCC-4 or TLCC-5 polypeptide has an amino acid sequence shown in SEQ ID NO:2 or 5. In other embodiments, the TLCC-4 or TLCC-5 polypeptide is substantially identical to SEQ ID NO:2 or 5, and retains the functional activity of the polypeptide of SEQ ID NO:2 or 5, yet differs in amino acid sequence due to natural allelic variation or mutagenesis, as described in detail in subsection I above. In another embodiment, the TLCC-4 or TLCC-5 polypeptide is a polypeptide which comprises an amino acid sequence at least about 50%, 55%, 60%, 65%, 70%, 75%, 80%, 85%, 90%, 95%, 96%, 97%, 98%, 99% or more identical to SEQ ID NO:2 or 5.

In another embodiment, the invention features a TLCC-4 or TLCC-5 polypeptide which is encoded by a nucleic acid molecule consisting of a nucleotide sequence at least about 50%, 55%, 60%, 65%, 70%, 75%, 80%, 85%, 90%, 95%, 96%, 97%, 98%, 99% or more identical to a nucleotide sequence of SEQ ID NO:1, 3, 4, or 6, or a complement thereof. This invention further features a TLCC-4 or TLCC-5 polypeptide which is encoded by a nucleic acid molecule consisting of a nucleotide sequence which hybridizes under stringent hybridization conditions to a complement of a nucleic acid molecule comprising the nucleotide sequence of SEQ ID NO:1, 3, 4, or 6, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the TLCC-4 amino acid sequence of SEQ ID NO:2 having 742 amino acid residues, at least 222, preferably at least 296, more preferably at least 371, more preferably at least 445, even more preferably at least 519, and even more preferably at least 593 or 667 or more amino acid residues are aligned; when aligning a second sequence to the TLCC-5 amino acid sequence of SEQ ID NO:5 having 1013 amino acid residues, at least 304, preferably at least 405, more preferably at least 506, more preferably at least 608, even more preferably at least 709, and even more preferably at least 810 or 912 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at the GCG website), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at the GCG website), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0 or version 2.0U), using a PAM120 weight residue table, a gap length penalty of 12 and a gap penalty of 4.

The nucleic acid and polypeptide sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TLCC-4 or TLCC-5 nucleic acid molecules of the invention. BLAST protein searches can be performed with the XBLAST program, score=100, wordlength=3, and a Blosum62 matrix to obtain amino acid sequences homologous to TLCC-4 or TLCC-5 polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the NIH website.

The invention also provides TLCC-4 or TLCC-5 chimeric or fusion proteins. As used herein, a TLCC-4 or TLCC-5 “chimeric protein” or “fusion protein” comprises a TLCC-4 or TLCC-5 polypeptide operatively linked to a non-TLCC-4 or non-TLCC-5 polypeptide. A “TLCC-4 or TLCC-5 polypeptide” refers to a polypeptide having an amino acid sequence corresponding to TLCC-4 or TLCC-5, whereas a “non-TLCC-4 or non-TLCC-5 polypeptide” refers to a polypeptide having an amino acid sequence corresponding to a protein which is not substantially homologous to the TLCC-4 or TLCC-5 polypeptide, e.g., a protein which is different from the TLCC-4 or TLCC-5 polypeptide and which is derived from the same or a different organism. Within a TLCC-4 or TLCC-5 fusion protein the TLCC-4 or TLCC-5 polypeptide can correspond to all or a portion of a TLCC-4 or TLCC-5 polypeptide. In a preferred embodiment, a TLCC-4 or TLCC-5 fusion protein comprises at least one biologically active portion of a TLCC-4 or TLCC-5 polypeptide. In another preferred embodiment, a TLCC-4 or TLCC-5 fusion protein comprises at least two biologically active portions of a TLCC-4 or TLCC-5 polypeptide. Within the fusion protein, the term “operatively linked” is intended to indicate that the TLCC-4 or TLCC-5 polypeptide and the non-TLCC-4 or non-TLCC-5 polypeptide are fused in-frame to each other. The non-TLCC-4 or non-TLCC-5 polypeptide can be fused to the N-terminus or C-terminus of the TLCC-4 or TLCC-5 polypeptide.

For example, in one embodiment, the fusion protein is a GST-TLCC-4 or GST-TLCC-5 fusion protein in which the TLCC-4 or TLCC-5 sequences are fused to the C-terminus of the GST sequences. Such fusion proteins can facilitate the purification of recombinant TLCC-4 or TLCC-5.

In another embodiment, the fusion protein is a TLCC-4 or TLCC-5 polypeptide containing a heterologous signal sequence at its N-terminus. In certain host cells (e.g., mammalian host cells), expression and/or secretion of TLCC-4 or TLCC-5 can be increased through the use of a heterologous signal sequence.

The TLCC-4 or TLCC-5 fusion proteins of the invention can be incorporated into pharmaceutical compositions and administered to a subject in vivo. The TLCC-4 or TLCC-5 fusion proteins can be used to affect the bioavailability of a TLCC-4 or TLCC-5 substrate. Use of TLCC-4 or TLCC-5 fusion proteins may be useful therapeutically for the treatment of disorders caused by, for example, (i) aberrant modification or mutation of a gene encoding a TLCC-4 or TLCC-5 polypeptide; (ii) mis-regulation of the TLCC-4 or TLCC-5 gene; and (iii) aberrant post-translational modification of a TLCC-4 or TLCC-5 polypeptide.

Moreover, the TLCC-4-fusion proteins or TLCC-5-fusion proteins of the invention can be used as immunogens to produce anti-TLCC-4 or anti-TLCC-5 antibodies in a subject, to purify TLCC-4 or TLCC-5 ligands and in screening assays to identify molecules which inhibit the interaction of TLCC-4 or TLCC-5 with a TLCC-4 or TLCC-5 substrate.

Preferably, a TLCC-4 or TLCC-5 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al John Wiley & Sons: 1992). Moreover, many expression vectors are commercially available that already encode a fusion moiety (e.g., a GST polypeptide). A TLCC-4-encoding or TLCC-5-encoding nucleic acid can be cloned into such an expression vector such that the fusion moiety is linked in-frame to the TLCC-4 or TLCC-5 polypeptide.

The present invention also pertains to variants of the TLCC-4 or TLCC-5 polypeptides which function as either TLCC-4 or TLCC-5 agonists (mimetics) or as TLCC-4 or TLCC-5 antagonists. Variants of the TLCC-4 or TLCC-5 polypeptides can be generated by mutagenesis, e.g., discrete point mutation or truncation of a TLCC-4 or TLCC-5 polypeptide. An agonist of the TLCC-4 or TLCC-5 polypeptides can retain substantially the same, or a subset, of the biological activities of the naturally occurring form of a TLCC-4 or TLCC-5 polypeptide. An antagonist of a TLCC-4 or TLCC-5 polypeptide can inhibit one or more of the activities of the naturally occurring form of the TLCC-4 or TLCC-5 polypeptide by, for example, competitively modulating a TLCC-4-mediated activity or TLCC-5-mediated activity of a TLCC-4 or TLCC-5 polypeptide. Thus, specific biological effects can be elicited by treatment with a variant of limited function. In one embodiment, treatment of a subject with a variant having a subset of the biological activities of the naturally occurring form of the polypeptide has fewer side effects in a subject relative to treatment with the naturally occurring form of the TLCC-4 or TLCC-5 polypeptide.

In one embodiment, variants of a TLCC-4 or TLCC-5 polypeptide which function as either TLCC-4 or TLCC-5 agonists (mimetics) or as TLCC-4 or TLCC-5 antagonists can be identified by screening combinatorial libraries of mutants, e.g., truncation mutants, of a TLCC-4 or TLCC-5 polypeptide for TLCC-4 or TLCC-5 polypeptide agonist or antagonist activity. In one embodiment, a variegated library of TLCC-4 or TLCC-5 variants is generated by combinatorial mutagenesis at the nucleic acid level and is encoded by a variegated gene library. A variegated library of TLCC-4 or TLCC-5 variants can be produced by, for example, enzymatically ligating a mixture of synthetic oligonucleotides into gene sequences such that a degenerate set of potential TLCC-4 or TLCC-5 sequences is expressible as individual polypeptides, or alternatively, as a set of larger fusion proteins (e.g., for phage display) containing the set of TLCC-4 or TLCC-5 sequences therein. There are a variety of methods which can be used to produce libraries of potential TLCC-4 or TLCC-5 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TLCC-4 or TLCC-5 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a TLCC-4 or TLCC-5 polypeptide coding sequence can be used to generate a variegated population of TLCC-4 or TLCC-5 fragments for screening and subsequent selection of variants of a TLCC-4 or TLCC-5 polypeptide. In one embodiment, a library of coding sequence fragments can be generated by treating a double stranded PCR fragment of a TLCC-4 or TLCC-5 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TLCC-4 or TLCC-5 polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TLCC-4 or TLCC-5 polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TLCC-4 or TLCC-5 variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated TLCC-4 or TLCC-5 library. For example, a library of expression vectors can be transfected into a cell line, e.g., an endothelial cell line, which ordinarily responds to TLCC-4 or TLCC-5 in a particular TLCC-4 or TLCC-5 substrate-dependent manner. The transfected cells are then contacted with TLCC-4 or TLCC-5 and the effect of expression of the mutant on signaling by the TLCC-4 or TLCC-5 substrate can be detected, e.g., by monitoring intracellular calcium, IP3, or diacylglycerol concentration, phosphorylation profile of intracellular proteins, or the activity of a TLCC-4-regulated or TLCC-5-regulated transcription factor. Plasmid DNA can then be recovered from the cells which score for inhibition, or alternatively, potentiation of signaling by the TLCC-4 or TLCC-5 substrate, and the individual clones further characterized.

An isolated TLCC-4 or TLCC-5 polypeptide, or a portion or fragment thereof, can be used as an immunogen to generate antibodies that bind TLCC-4 or TLCC-5 using standard techniques for polyclonal and monoclonal antibody preparation. A full-length TLCC-4 or TLCC-5 polypeptide can be used or, alternatively, the invention provides antigenic peptide fragments of TLCC-4 or TLCC-5 for use as immunogens. The antigenic peptide of TLCC-4 or TLCC-5 comprises at least 8 amino acid residues of the amino acid sequence shown in SEQ ID NO:2 or 5 and encompasses an epitope of TLCC-4 or TLCC-5 such that an antibody raised against the peptide forms a specific immune complex with TLCC-4 or TLCC-5. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of TLCC-4 or TLCC-5 that are located on the surface of the polypeptide, e.g., hydrophilic regions, as well as regions with high antigenicity (see, for example, FIGS. 4 and 7).

A TLCC-4 or TLCC-5 immunogen typically is used to prepare antibodies by immunizing a suitable subject, (e.g., rabbit, goat, mouse or other mammal) with the immunogen. An appropriate immunogenic preparation can contain, for example, recombinantly expressed TLCC-4 or TLCC-5 polypeptide or a chemically synthesized TLCC-4 or TLCC-5 polypeptide. The preparation can further include an adjuvant, such as Freund's complete or incomplete adjuvant, or similar immunostimulatory agent. Immunization of a suitable subject with an immunogenic TLCC-4 or TLCC-5 preparation induces a polyclonal anti-TLCC-4 or anti-TLCC-5 antibody response.

Accordingly, another aspect of the invention pertains to anti-TLCC-4 or anti-TLCC-5 antibodies. The term “antibody” as used herein refers to immunoglobulin molecules and immunologically active portions of immunoglobulin molecules, i.e., molecules that contain an antigen binding site which specifically binds (immunoreacts with) an antigen, such as TLCC-4 or TLCC-5. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind TLCC-4 or TLCC-5. The term “monoclonal antibody” or “monoclonal antibody composition”, as used herein, refers to a population of antibody molecules that contain only one species of an antigen binding site capable of immunoreacting with a particular epitope of TLCC-4 or TLCC-5. A monoclonal antibody composition thus typically displays a single binding affinity for a particular TLCC-4 or TLCC-5 polypeptide with which it immunoreacts.

Polyclonal anti-TLCC-4 or anti-TLCC-5 antibodies can be prepared as described above by immunizing a suitable subject with a TLCC-4 or TLCC-5 immunogen. The anti-TLCC-4 or anti-TLCC-5 antibody titer in the immunized subject can be monitored over time by standard techniques, such as with an enzyme linked immunosorbent assay (ELISA) using immobilized TLCC-4 or TLCC-5. If desired, the antibody molecules directed against TLCC-4 or TLCC-5 can be isolated from the mammal (e.g., from the blood) and further purified by well known techniques, such as protein A chromatography to obtain the IgG fraction. At an appropriate time after immunization, e.g., when the anti-TLCC-4 or anti-TLCC-5 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see, generally, R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a TLCC-4 or TLCC-5 immunogen as described above, and the culture supernatants of the resulting hybridoma cells are screened to identify a hybridoma producing a monoclonal antibody that binds TLCC-4 or TLCC-5.

Any of the many well known protocols used for fusing lymphocytes and immortalized cell lines can be applied for the purpose of generating an anti-TLCC-4 or anti-TLCC-5 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TLCC-4 or TLCC-5, e.g., using a standard ELISA assay.

Alternative to preparing monoclonal antibody-secreting hybridomas, a monoclonal anti-TLCC-4 or anti-TLCC-5 antibody can be identified and isolated by screening a recombinant combinatorial immunoglobulin library (e.g., an antibody phage display library) with TLCC-4 or TLCC-5 to thereby isolate immunoglobulin library members that bind TLCC-4 or TLCC-5. Kits for generating and screening phage display libraries are commercially available (e.g., the Pharmacia Recombinant Phage Antibody System, Catalog No. 27-9400-01; and the Stratagene SurjZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-TLCC-4 or anti-TLCC-5 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-TLCC-4 or anti-TLCC-5 antibody (e.g., monoclonal antibody) can be used to isolate TLCC-4 or TLCC-5 by standard techniques, such as affinity chromatography or immunoprecipitation. An anti-TLCC-4 or anti-TLCC-5 antibody can facilitate the purification of natural TLCC-4 or TLCC-5 from cells and of recombinantly produced TLCC-4 or TLCC-5 expressed in host cells. Moreover, an anti-TLCC-4 or anti-TLCC-5 antibody can be used to detect TLCC-4 or TLCC-5 polypeptide (e.g., in a cellular lysate or cell supernatant) in order to evaluate the abundance and pattern of expression of the TLCC-4 or TLCC-5 polypeptide. Anti-TLCC-4 or anti-TLCC-5 antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, for example recombinant expression vectors, containing a TLCC-4 or TLCC-5 nucleic acid molecule or vectors containing a nucleic acid molecule which encodes a TLCC-4 or TLCC-5 polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors” . In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., TLCC-4 or TLCC-5 polypeptides, mutant forms of TLCC-4 or TLCC-5 polypeptides, fusion proteins, and the like).

Accordingly, an exemplary embodiment provides a method for producing a protein, preferably a TLCC-4 or TLCC-5 polypeptide, by culturing in a suitable medium a host cell of the invention (e.g., a mammalian host cell such as a non-human mammalian cell) containing a recombinant expression vector, such that the protein is produced.

The recombinant expression vectors of the invention can be designed for expression of TLCC-4 or TLCC-5 polypeptides in prokaryotic or eukaryotic cells. For example, TLCC-4 or TLCC-5 polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in TLCC-4 or TLCC-5 activity assays, (e.g., direct assays or competitive assays described in detail below), or to generate antibodies specific for TLCC-4 or TLCC-5 polypeptides, for example. In a preferred embodiment, a TLCC-4 or TLCC-5 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology. Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the TLCC-4 or TLCC-5 expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, TLCC-4 or TLCC-5 polypeptides can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the cc-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TLCC-4 or TLCC-5 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a TLCC-4 or TLCC-5 nucleic acid molecule of the invention is introduced, e.g., a TLCC-4 or TLCC-5 nucleic acid molecule within a vector (e.g., a recombinant expression vector) or a TLCC-4 or TLCC-5 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TLCC-4 or TLCC-5 polypeptide can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a TLCC-4 or TLCC-5 polypeptide, or a nucleic acid encoding a selectable marker can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a TLCC-4 or TLCC-5 polypeptide. Accordingly, the invention further provides methods for producing a TLCC-4 or TLCC-5 polypeptide using the host cells of the invention. In one embodiment, the method comprises culturing the host cell of the invention (into which a recombinant expression vector encoding a TLCC-4 or TLCC-5 polypeptide has been introduced) in a suitable medium such that a TLCC-4 or TLCC-5 polypeptide is produced. In another embodiment, the method further comprises isolating a TLCC-4 or TLCC-5 polypeptide from the medium or the host cell.

The host cells of the invention can also be used to produce non-human transgenic animals. For example, in one embodiment, a host cell of the invention is a fertilized oocyte or an embryonic stem cell into which TLCC-4-coding sequences or TLCC-5-coding sequences have been introduced. Such host cells can then be used to create non-human transgenic animals in which exogenous TLCC-4 or TLCC-5 sequences have been introduced into their genome or homologous recombinant animals in which endogenous TLCC-4 or TLCC-5 sequences have been altered. Such animals are useful for studying the function and/or activity of a TLCC-4 or TLCC-5 and for identifying and/or evaluating modulators of TLCC-4 or TLCC-5 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TLCC-4 or TLCC-5 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a TLCC-4-encoding nucleic acid or TLCC-5-encoding nucleic acid into the male pronuclei of a fertilized oocyte, e.g., by microinjection, retroviral infection, and allowing the oocyte to develop in a pseudopregnant female foster animal. The TLCC-4 or TLCC-5 cDNA sequence of SEQ ID NO:1 or 4 can be introduced as a transgene into the genome of a non-human animal. Alternatively, a nonhuman homologue of a human TLCC-4 or TLCC-5 gene, such as a mouse or rat TLCC-4 or TLCC-5 gene, can be used as a transgene. Alternatively, a TLCC-4 or TLCC-5 gene homologue, such as another TLCC-4 or TLCC-5 family member, can be isolated based on hybridization to the TLCC-4 or TLCC-5 cDNA sequences of SEQ ID NO:1, 3, 4, or 6, or the DNA inserts of the plasmids deposited with ATCC as Accession Numbers______ (described further in subsection I above) and used as a transgene. Intronic sequences and polyadenylation signals can also be included in the transgene to increase the efficiency of expression of the transgene. A tissue-specific regulatory sequence(s) can be operably linked to a TLCC-4 or TLCC-5 transgene to direct expression of a TLCC-4 or TLCC-5 polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a TLCC-4 or TLCC-5 transgene in its genome and/or expression of TLCC-4 or TLCC-5 mRNA in tissues or cells of the animals. A transgenic founder animal can then be used to breed additional animals carrying the transgene. Moreover, transgenic animals carrying a transgene encoding a TLCC-4 or TLCC-5 polypeptide can further be bred to other transgenic animals carrying other transgenes.

To create a homologous recombinant animal, a vector is prepared which contains at least a portion of a TLCC-4 or TLCC-5 gene into which a deletion, addition or substitution has been introduced to thereby alter, e.g., functionally disrupt, the TLCC-4 or TLCC-5 gene. The TLCC-4 or TLCC-5 gene can be a human gene (e.g., the cDNA of SEQ ID NO:3 or 6), but more preferably, is a non-human homologue of a human TLCC-4 or TLCC-5 gene (e.g., a cDNA isolated by stringent hybridization with the nucleotide sequence of SEQ ID NO:1 or 4). For example, a mouse TLCC-4 or TLCC-5 gene can be used to construct a homologous recombination nucleic acid molecule, e.g., a vector, suitable for altering an endogenous TLCC-4 or TLCC-5 gene in the mouse genome. In a preferred embodiment, the homologous recombination nucleic acid molecule is designed such that, upon homologous recombination, the endogenous TLCC-4 or TLCC-5 gene is functionally disrupted (i.e., no longer encodes a functional polypeptide; also referred to as a “knock out” vector). Alternatively, the homologous recombination nucleic acid molecule can be designed such that, upon homologous recombination, the endogenous TLCC-4 or TLCC-5 gene is mutated or otherwise altered but still encodes functional polypeptide (e.g., the upstream regulatory region can be altered to thereby alter the expression of the endogenous TLCC-4 or TLCC-5 polypeptide). In the homologous recombination nucleic acid molecule, the altered portion of the TLCC-4 or TLCC-5 gene is flanked at its 5′ and 3′ ends by additional nucleic acid sequence of the TLCC-4 or TLCC-5 gene to allow for homologous recombination to occur between the exogenous TLCC-4 or TLCC-5 gene carried by the homologous recombination nucleic acid molecule and an endogenous TLCC-4 or TLCC-5 gene in a cell, e.g., an embryonic stem cell. The additional flanking TLCC-4 or TLCC-5 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced TLCC-4 or TLCC-5 gene has homologously recombined with the endogenous TLCC-4 or TLCC-5 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TLCC-4 and TLCC-5 nucleic acid molecules, fragments of TLCC-4 and TLCC-5 polypeptides, and anti-TLCC-4 and anti-TLCC-5 antibodies (also referred to herein as “active compounds” ) of the invention can be incorporated into pharmaceutical compositions suitable for administration. Such compositions typically comprise the nucleic acid molecule, polypeptide, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g. a fragment of a TLCC-4 or TLCC-5 polypeptide or an anti-TLCC-4 or anti-TLCC-5 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between about 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a polypeptide possessing a desired biological activity. Such polypeptides may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470), or by stereotactic injection (see, e.g., Chen et al. (1994) Proc. Natl. Acad. Sci. USA 91 :3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, polypeptides, polypeptide homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a TLCC-4 or TLCC-5 polypeptide of the invention has one or more of the following activities: (1) modulates membrane excitability, (2) influences the resting potential of membranes, (3) modulates wave forms and frequencies of action potentials, (4) modulates thresholds of excitation, (5) modulates neurite outgrowth and synaptogenesis, (6) modulates signal transduction, and (7) participates in nociception.

The isolated nucleic acid molecules of the invention can be used, for example, to express a TLCC-4 or TLCC-5 polypeptide (e.g., via a recombinant expression vector in a host cell in gene therapy applications), to detect TLCC-4 or TLCC-5 mRNA (e.g., in a biological sample) or a genetic alteration in a TLCC-4 or TLCC-5 gene, and to modulate TLCC-4 or TLCC-5 activity, as described further below. The TLCC-4 or TLCC-5 polypeptides can be used to treat disorders characterized by insufficient or excessive production of a TLCC-4 or TLCC-5 substrate or production of TLCC-4 or TLCC-5 inhibitors. In addition, the TLCC-4 or TLCC-5 polypeptides can be used to screen for naturally occurring TLCC-4 or TLCC-5 substrates, to screen for drugs or compounds which modulate TLCC-4 or TLCC-5 activity, as well as to treat disorders characterized by insufficient or excessive production of TLCC-4 or TLCC-5 polypeptide or production of TLCC-4 or TLCC-5 polypeptide forms which have decreased, aberrant or unwanted activity compared to TLCC-4 or TLCC-5 wild type polypeptide (e.g., CNS disorders (such as neurodegenerative disorders), pain disorders, or cellular growth, differentiation, or migration disorders). Moreover, the anti-TLCC-4 or anti-TLCC-5 antibodies of the invention can be used to detect and isolate TLCC-4 or TLCC-5 polypeptides, to regulate the bioavailability of TLCC-4 or TLCC-5 polypeptides, and modulate TLCC-4 or TLCC-5 activity.

A. Screening Assays

The invention provides a method (also referred to herein as a “screening assay”) for identifying modulators, i.e., candidate or test compounds or agents (e.g., peptides, peptidomimetics, small molecules or other drugs) which bind to TLCC-4 or TLCC-5 polypeptides, have a stimulatory or inhibitory effect on, for example, TLCC-4 or TLCC-5 expression or TLCC-4 or TLCC-5 activity, or have a stimulatory or inhibitory effect on, for example, the expression or activity of TLCC-4 or TLCC-5 substrate.

In one embodiment, the invention provides assays for screening candidate or test compounds which are substrates of a TLCC-4 or TLCC-5 polypeptide or biologically active portion thereof. In another embodiment, the invention provides assays for screening candidate or test compounds which bind to or modulate the activity of a TLCC-4 or TLCC-5 polypeptide or polypeptide or biologically active portion thereof.

The screening assays of the present invention include, but are not limited to, screening assays specific for calcium receptors, e.g., measuring intracellular Ca²⁺ concentrations, and/or assaying responses to the presence of the calcium channel stimulators inositol-1,4,5-triphosphate (IP₃) and/or diacylglycerol (DAG) (see, e.g., Hofmann et al (1999) Nature 397:259-263; Okada et al. (1999) J. Biol. Chem. 274:27359-27370; Hofmann et al. (2000) J. Mol Med. 78:14-25). Intracellular Ca²⁺ levels may be assayed, for example, using the fluorescent dye fura-2 as an indicator. Stimulation by DAG may be assayed, for example, using DAG analogues (e.g., OAG (1-oleoyl-2-acetyl-sn-glycerol) and DOG (1,2-dioctanoyl-sn-glycerol)). IP₃ stimulation may be analyzed, for example, using the PLC inhibitor U-73122 in the presence of ATP, which suppresses IP₃-dependent Ca²⁺ release.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a TLCC-4 or TLCC-5 polypeptide or biologically active portion thereof is contacted with a test compound and the ability of the test compound to modulate TLCC-4 or TLCC-5 activity is determined. Determining the ability of the test compound to modulate TLCC-4 or TLCC-5 activity can be accomplished by monitoring, for example, intracellular calcium, IP3, or diacylglycerol concentration, phosphorylation profile of intracellular proteins, or the activity of a TLCC-4-regulated or TLCC-5-regulated transcription factor. The cell, for example, can be of mammalian origin, e.g., a neuronal cell, skin cell, or a liver cell.

The ability of the test compound to modulate TLCC-4 or TLCC-5 binding to a substrate or to bind to TLCC-4 or TLCC-5 can also be determined. Determining the ability of the test compound to modulate TLCC-4 or TLCC-5 binding to a substrate can be accomplished, for example, by coupling the TLCC-4 or TLCC-5 substrate with a radioisotope or enzymatic label such that binding of the TLCC-4 or TLCC-5 substrate to TLCC-4 or TLCC-5 can be determined by detecting the labeled TLCC-4 or TLCC-5 substrate in a complex. Alternatively, TLCC-4 or TLCC-5 could be coupled with a radioisotope or enzymatic label to monitor the ability of a test compound to modulate TLCC-4 or TLCC-5 binding to a TLCC-4 or TLCC-5 substrate in a complex. Determining the ability of the test compound to bind TLCC-4 or TLCC-5 can be accomplished, for example, by coupling the compound with a radioisotope or enzymatic label such that binding of the compound to TLCC-4 or TLCC-5 can be determined by detecting the labeled TLCC-4 or TLCC-5 compound in a complex. For example, compounds (e.g., TLCC-4 or TLCC-5 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a TLCC-4 or TLCC-5 substrate) to interact with TLCC-4 or TLCC-5 without the labeling of any of the interactants. For example, a microphysiometer can be used to detect the interaction of a compound with TLCC-4 or TLCC-5 without the labeling of either the compound or the TLCC-4 or TLCC-5. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and TLCC-4 or TLCC-5.

In another embodiment, an assay is a cell-based assay comprising contacting a cell expressing a TLCC-4 or TLCC-5 target molecule (e.g., a TLCC-4 or TLCC-5 substrate) with a test compound and determining the ability of the test compound to modulate (e.g., stimulate or inhibit) the activity of the TLCC-4 or TLCC-5 target molecule. Determining the ability of the test compound to modulate the activity of a TLCC-4 or TLCC-5 target molecule can be accomplished, for example, by determining the ability of the TLCC-4 or TLCC-5 polypeptide to bind to or interact with the TLCC-4 or TLCC-5 target molecule.

Determining the ability of the TLCC-4 or TLCC-5 polypeptide, or a biologically active fragment thereof, to bind to or interact with a TLCC-4 or TLCC-5 target molecule can be accomplished by one of the methods described above for determining direct binding. In a preferred embodiment, determining the ability of the TLCC-4 or TLCC-5 polypeptide to bind to or interact with a TLCC-4 or TLCC-5 target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a TLCC-4 or TLCC-5 polypeptide or biologically active portion thereof is contacted with a test compound and the ability of the test compound to bind to the TLCC-4 or TLCC-5 polypeptide or biologically active portion thereof is determined. Preferred biologically active portions of the TLCC-4 or TLCC-5 polypeptides to be used in assays of the present invention include fragments which participate in interactions with non-TLCC-4 or non- TLCC-5 molecules, e.g., fragments with high surface probability scores (see, for example, FIGS. 4 and 7). Binding of the test compound to the TLCC-4 or TLCC-5 polypeptide can be determined either directly or indirectly as described above. In a preferred embodiment, the assay includes contacting the TLCC-4 or TLCC-5 polypeptide or biologically active portion thereof with a known compound which binds TLCC-4 or TLCC-5 to form an assay mixture, contacting the assay mixture with a test compound, and determining the ability of the test compound to interact with a TLCC-4 or TLCC-5 polypeptide, wherein determining the ability of the test compound to interact with a TLCC-4 or TLCC-5 polypeptide comprises determining the ability of the test compound to preferentially bind to TLCC-4 or TLCC-5 or biologically active portion thereof as compared to the known compound.

In another embodiment, the assay is a cell-free assay in which a TLCC-4 or TLCC-5 polypeptide or biologically active portion thereof is contacted with a test compound and the ability of the test compound to modulate (e.g., stimulate or inhibit) the activity of the TLCC-4 or TLCC-5 polypeptide or biologically active portion thereof is determined. Determining the ability of the test compound to modulate the activity of a TLCC-4 or TLCC-5 polypeptide can be accomplished, for example, by determining the ability of the TLCC-4 or TLCC-5 polypeptide to bind to a TLCC-4 or TLCC-5 target molecule by one of the methods described above for determining direct binding. Determining the ability of the TLCC-4 or TLCC-5 polypeptide to bind to a TLCC-4 or TLCC-5 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a TLCC-4 or TLCC-5 polypeptide can be accomplished by determining the ability of the TLCC-4 or TLCC-5 polypeptide to further modulate the activity of a downstream effector of a TLCC-4 or TLCC-5 target molecule. For example, the activity of the effector molecule on an appropriate target can be determined or the binding of the effector to an appropriate target can be determined as previously described.

In yet another embodiment, the cell-free assay involves contacting a TLCC-4 or TLCC-5 polypeptide or biologically active portion thereof with a known compound which binds the TLCC-4 or TLCC-5 polypeptide to form an assay mixture, contacting the assay mixture with a test compound, and determining the ability of the test compound to interact with the TLCC-4 or TLCC-5 polypeptide, wherein determining the ability of the test compound to interact with the TLCC-4 or TLCC-5 polypeptide comprises determining the ability of the TLCC-4 or TLCC-5 polypeptide to preferentially bind to or modulate the activity of a TLCC-4 or TLCC-5 target molecule.

In more than one embodiment of the above assay methods of the present invention, it may be desirable to immobilize either TLCC-4 or TLCC-5 or its target molecule to facilitate separation of complexed from uncomplexed forms of one or both of the polypeptides, as well as to accommodate automation of the assay. Binding of a test compound to a TLCC-4 or TLCC-5 polypeptide, or interaction of a TLCC-4 or TLCC-5 polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/ TLCC-4 or TLCC-5 fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or TLCC-4 or TLCC-5 polypeptide, and the mixture incubated under conditions conducive to complex formation (e.g., at physiological conditions for salt and pH). Following incubation, the beads or micrometer plate wells are washed to remove any unbound components, the matrix immobilized in the case of beads, complex determined either directly or indirectly, for example, as described above. Alternatively, the complexes can be dissociated from the matrix, and the level of TLCC-4 or TLCC-5 binding or activity determined using standard techniques.

Other techniques for immobilizing proteins on matrices can also be used in the screening assays of the invention. For example, either a TLCC-4 or TLCC-5 polypeptide or a TLCC-4 or TLCC-5 target molecule can be immobilized utilizing conjugation of biotin and streptavidin. Biotinylated TLCC-4 or TLCC-5 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with TLCC-4 or TLCC-5 polypeptide or target molecules but which do not interfere with binding of the TLCC-4 or TLCC-5 polypeptide to its target molecule can be derivatized to the wells of the plate, and unbound target or TLCC-4 or TLCC-5 polypeptide trapped in the wells by antibody conjugation. Methods for detecting such complexes, in addition to those described above for the GST-immobilized complexes, include immunodetection of complexes using antibodies reactive with the TLCC-4 or TLCC-5 polypeptide or target molecule, as well as enzyme-linked assays which rely on detecting an enzymatic activity associated with the TLCC-4 or TLCC-5 polypeptide or target molecule.

In another embodiment, modulators of TLCC-4 or TLCC-5 expression are identified in a method wherein a cell is contacted with a candidate compound and the expression of TLCC-4 or TLCC-5 mRNA or polypeptide in the cell is determined. The level of expression of TLCC-4 or TLCC-5 mRNA or polypeptide in the presence of the candidate compound is compared to the level of expression of TLCC-4 or TLCC-5 mRNA or polypeptide in the absence of the candidate compound. The candidate compound can then be identified as a modulator of TLCC-4 or TLCC-5 expression based on this comparison. For example, when expression of TLCC-4 or TLCC-5 mRNA or polypeptide is greater (statistically significantly greater) in the presence of the candidate compound than in its absence, the candidate compound is identified as a stimulator of TLCC-4 or TLCC-5 mRNA or polypeptide expression. Alternatively, when expression of TLCC-4 or TLCC-5 mRNA or polypeptide is less (statistically significantly less) in the presence of the candidate compound than in its absence, the candidate compound is identified as an inhibitor of TLCC-4 or TLCC-5 mRNA or polypeptide expression. The level of TLCC-4 or TLCC-5 mRNA or polypeptide expression in the cells can be determined by methods described herein for detecting TLCC-4 or TLCC-5 mRNA or polypeptide.

In yet another aspect of the invention, the TLCC-4 or TLCC-5 polypeptides can be used as “bait proteins” in a two-hybrid assay or three-hybrid assay (see, e.g., U.S. Pat. No. 5,283,317; Zervos et al. (1993) Cell 72:223-232; Madura et al (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with TLCC-4 or TLCC-5 (“TLCC-4-binding proteins or TLCC-5-binding proteins” or “TLCC-4-bp or TLCC-5-bp”) and are involved in TLCC-4 or TLCC-5 activity. Such TLCC-4-binding proteins or TLCC-5-binding proteins are also likely to be involved in the propagation of signals by the TLCC-4 or TLCC-5 polypeptides or TLCC-4 or TLCC-5 targets as, for example, downstream elements of a TLCC-4-mediated or TLCC-5-mediated signaling pathway. Alternatively, such TLCC-4-binding proteins or TLCC-5-binding proteins are likely to be TLCC-4 or TLCC-5 inhibitors.

The two-hybrid system is based on the modular nature of most transcription factors, which consist of separable DNA-binding and activation domains. Briefly, the assay utilizes two different DNA constructs. In one construct, the gene that codes for a TLCC-4 or TLCC-5 polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TLCC-4-dependent or TLCC-5-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TLCC-4 or TLCC-5 polypeptide.

In another aspect, the invention pertains to a combination of two or more of the assays described herein. For example, a modulating agent can be identified using a cell-based or a cell free assay, and the ability of the agent to modulate the activity of a TLCC-4 or TLCC-5 polypeptide can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a TLCC-4 or TLCC-5 modulating agent, an antisense TLCC-4 or TLCC-5 nucleic acid molecule, a TLCC-4-specific or TLCC-5-specific antibody, or a TLCC-4-binding partner or TLCC-5-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Models for studying pain in vivo include rat models of neuropathic pain caused by methods such as intraperitoneal administration of Taxol (Authier et al. (2000) Brain Res. 887:239-249), chronic constriction injury (CCI), partial sciatic nerve transection (Linenlaub and Sommer (2000) Pain 89:97-106), transection of the tibial and sural nerves (Lee et al. (2000) Neurosci. Lett. 291:29-32), the spared nerve injury model (Decosterd and Woolf (2000) Pain 87:149-158), cuffing the sciatic nerve (Pitcher and Henry (2000) Eur. J. Neurosci. 12:2006-2020), unilateral tight ligation (Esser and Sawynok (2000) Eur. J. Pharmacol. 399:131-139), L5 spinal nerve ligation (Honroe et al. (2000) Neurosci. 98:585-598), and photochemically induced ischemic nerve injury (Hao et al. (2000) Exp. Neurol. 163:231-238); rat models of nociceptive pain caused by methods such as the Chung Method, the Bennett Method, and intraperitoneal administration of Complete Freund's adjuvant (Abdi et al. (2000) Anesth. Analg. 91:955-959); rat models of post-incisional pain caused by incising the skin and fascia of a hind paw (Olivera and Prado (2000) Braz. J. Med. Biol. Res. 33:957-960); rat models of cancer pain caused by methods such as injecting osteolytic sarcoma cells into the femur (Honroe et al. (2000) Neurosci. 98:585-598); and rat models of visceral pain caused by methods such as intraperitoneal administration of cyclophosphamide.

Various methods of determining an animal's response to pain are known in the art. Examples of such methods include, but are not limited to brief intense exposure to a focused heat source, administration of a noxious chemical subcutaneously, the tail flick test, the hot plate test, the formalin test, the Von Frey threshold test, and testing for stress-induced analgesia (e.g., by restraint, foot shock, and/or cold water swim) (Crawley (2000) What's Wrong With My Mouse? Wiley-Liss pp. 72-75).

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the TLCC-4 or TLCC-5 nucleotide sequences, described herein, can be used to map the location of the TLCC-4 or TLCC-5 genes on a chromosome. The mapping of the TLCC-4 or TLCC-5 sequences to chromosomes is an important first step in correlating these sequences with genes associated with disease.

Briefly, TLCC-4 or TLCC-5 genes can be mapped to chromosomes by preparing PCR primers (preferably 15-25 bp in length) from the TLCC-4 or TLCC-5 nucleotide sequences. Computer analysis of the TLCC-4 or TLCC-5 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the TLCC-4 or TLCC-5 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the TLCC-4 or TLCC-5 nucleotide sequences to design oligonucleotide primers, sublocalization can be achieved with panels of fragments from specific chromosomes. Other mapping strategies which can similarly be used to map a TLCC-4 or TLCC-5 sequence to its chromosome include in situ hybridization (described in Fan, Y. et al. (1990) Proc. Natl. Acad Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the TLCC-4 or TLCC-5 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The TLCC-4 or TLCC-5 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the TLCC-4 or TLCC-5 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The TLCC-4 or TLCC-5 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 or 4 can comfortably provide positive individual identification with a panel of perhaps 10 to 1,000 primers which each yield a noncoding amplified sequence of 100 bases. If predicted coding sequences, such as those in SEQ ID NO:3 or 6 are used, a more appropriate number of primers for positive individual identification would be 500-2,000.

If a panel of reagents from TLCC-4 or TLCC-5 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of TLCC-4 and TLCC-5 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 or 4 are particularly appropriate for this use as greater numbers of polymorphisms occur in the noncoding regions, making it easier to differentiate individuals using this technique. Examples of polynucleotide reagents include the TLCC-4 or TLCC-5 nucleotide sequences or portions thereof, e.g., fragments derived from the noncoding regions of SEQ ID NO: 1 or 4 having a length of at least 20 bases, preferably at least 30 bases.

The TLCC-4 or TLCC-5 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such TLCC-4 or TLCC-5 probes can be used to identify tissue by species and/or by organ type.

In a similar fashion, these reagents, e.g., TLCC-4 or TLCC-5 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining TLCC-4 or TLCC-5 polypeptide and/or nucleic acid expression as well as TLCC-4 or TLCC-5 activity, in the context of a biological sample (e.g., blood, serum, cells, tissue) to thereby determine whether an individual is afflicted with a disease or disorder, or is at risk of developing a disorder, associated with aberrant or unwanted TLCC-4 or TLCC-5 expression or activity. The invention also provides for prognostic (or predictive) assays for determining whether an individual is at risk of developing a disorder associated with TLCC-4 or TLCC-5 polypeptide, nucleic acid expression or activity. For example, mutations in a TLCC-4 or TLCC-5 gene can be assayed in a biological sample. Such assays can be used for prognostic or predictive purpose to thereby prophylactically treat an individual prior to the onset of a disorder characterized by or associated with TLCC-4 or TLCC-5 polypeptide, nucleic acid expression or activity.

Another aspect of the invention pertains to monitoring the influence of agents (e.g., drugs, compounds) on the expression or activity of TLCC-4 or TLCC-5 in clinical trials.

These and other agents are described in further detail in the following sections.

1. Diagnostic Assays

An exemplary method for detecting the presence or absence of TLCC-4 or TLCC-5 polypeptide or nucleic acid in a biological sample involves obtaining a biological sample from a test subject and contacting the biological sample with a compound or an agent capable of detecting TLCC-4 or TLCC-5 polypeptide or nucleic acid (e.g., mRNA, or genomic DNA) that encodes TLCC-4 or TLCC-5 polypeptide such that the presence of TLCC-4 or TLCC-5 polypeptide or nucleic acid is detected in the biological sample. In another aspect, the present invention provides a method for detecting the presence of TLCC-4 or TLCC-5 activity in a biological sample by contacting the biological sample with an agent capable of detecting an indicator of TLCC-4 or TLCC-5 activity such that the presence of TLCC-4 or TLCC-5 activity is detected in the biological sample. A preferred agent for detecting TLCC-4 or TLCC-5 mRNA or genomic DNA is a labeled nucleic acid probe capable of hybridizing to TLCC-4 or TLCC-5 mRNA or genomic DNA. The nucleic acid probe can be, for example, the TLCC-4 or TLCC-5 nucleic acid set forth in SEQ ID NO:1, 3, 4, or 6, or the DNA inserts of the plasmids deposited with ATCC as Accession Numbers______, or a portion thereof, such as an oligonucleotide of at least 15, 30, 50, 100, 250 or 500 nucleotides in length and sufficient to specifically hybridize under stringent conditions to TLCC-4 or TLCC-5 mRNA or genomic DNA. Other suitable probes for use in the diagnostic assays of the invention are described herein.

A preferred agent for detecting TLCC-4 or TLCC-5 polypeptide is an antibody capable of binding to TLCC-4 or TLCC-5 polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i. e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TLCC-4 or TLCC-5 mRNA, polypeptide, or genomic DNA in a biological sample in vitro as well as in vivo. For example, in vitro techniques for detection of TLCC-4 or TLCC-5 mRNA include Northern hybridizations and in situ hybridizations. In vitro techniques for detection of TLCC-4 or TLCC-5 polypeptide include enzyme linked immunosorbent assays (ELISAs), Western blots, immunoprecipitations and immunofluorescence. In vitro techniques for detection of TLCC-4 or TLCC-5 genomic DNA include Southern hybridizations. Furthermore, in vivo techniques for detection of TLCC-4 or TLCC-5 polypeptide include introducing into a subject a labeled anti-TLCC-4 or anti-TLCC-5 antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding a TLCC-4 or TLCC-5 polypeptide; (ii) aberrant expression of a gene encoding a TLCC-4 or TLCC-5 polypeptide; (iii) mis-regulation of the gene; and (iii) aberrant post-translational modification of a TLCC-4 or TLCC-5 polypeptide, wherein a wild-type form of the gene encodes a protein with a TLCC-4 or TLCC-5 activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains polypeptide molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting TLCC-4 or TLCC-5 polypeptide, mRNA, or genomic DNA, such that the presence of TLCC-4 or TLCC-5 polypeptide, mRNA or genomic DNA is detected in the biological sample, and comparing the presence of TLCC-4 or TLCC-5 polypeptide, mRNA or genomic DNA in the control sample with the presence of TLCC-4 or TLCC-5 polypeptide, mRNA or genomic DNA in the test sample.

The invention also encompasses kits for detecting the presence of TLCC-4 or TLCC-5 in a biological sample. For example, the kit can comprise a labeled compound or agent capable of detecting TLCC-4 or TLCC-5 polypeptide or mRNA in a biological sample; means for determining the amount of TLCC-4 or TLCC-5 in the sample; and means for comparing the amount of TLCC-4 or TLCC-5 in the sample with a standard. The compound or agent can be packaged in a suitable container. The kit can further comprise instructions for using the kit to detect TLCC-4 or TLCC-5 polypeptide or nucleic acid.

2. Prognostic Assays

The diagnostic methods described herein can furthermore be utilized to identify subjects having or at risk of developing a disease or disorder associated with aberrant or unwanted TLCC-4 or TLCC-5 expression or activity. As used herein, the term “aberrant” includes a TLCC-4 or TLCC-5 expression or activity which deviates from the wild type TLCC-4 or TLCC-5 expression or activity. Aberrant expression or activity includes increased or decreased expression or activity, as well as expression or activity which does not follow the wild type developmental pattern of expression or the subcellular pattern of expression. For example, aberrant TLCC-4 or TLCC-5 expression or activity is intended to include the cases in which a mutation in the TLCC-4 or TLCC-5 gene causes the TLCC-4 or TLCC-5 gene to be under-expressed or over-expressed and situations in which such mutations result in a non-functional TLCC-4 or TLCC-5 polypeptide or a polypeptide which does not function in a wild-type fashion, e.g., a polypeptide which does not interact with a TLCC-4 or TLCC-5 substrate, e.g., a non-calcium channel subunit or ligand and/or a non-vanilloid receptor subunit or ligand, or one which interacts with a non-TLCC-4 or non-TLCC-5 substrate, e.g. a non-calcium channel subunit or ligand and/or a non-vanilloid receptor subunit or ligand. As used herein, the term “unwanted” includes an unwanted phenomenon involved in a biological response, such as cellular proliferation. For example, the term unwanted includes a TLCC-4 or TLCC-5 expression or activity which is undesirable in a subject.

The assays described herein, such as the preceding diagnostic assays or the following assays, can be utilized to identify a subject having or at risk of developing a disorder associated with a misregulation in TLCC-4 or TLCC-5 polypeptide activity or nucleic acid expression, such as a CNS disorder (e.g., a neurodegenerative disorder, a pain disorder, or a cellular proliferation, growth, differentiation, or migration disorder). Alternatively, the prognostic assays can be utilized to identify a subject having or at risk for developing a disorder associated with a misregulation in TLCC-4 or TLCC-5 polypeptide activity or nucleic acid expression, such as a CNS disorder, a pain disorder, or a cellular proliferation, growth, differentiation, or migration disorder. Thus, the present invention provides a method for identifying a disease or disorder associated with aberrant or unwanted TLCC-4 or TLCC-5 expression or activity in which a test sample is obtained from a subject and TLCC-4 or TLCC-5 polypeptide or nucleic acid (e.g., mRNA or genomic DNA) is detected, wherein the presence of TLCC-4 or TLCC-5 polypeptide or nucleic acid is diagnostic for a subject having or at risk of developing a disease or disorder associated with aberrant or unwanted TLCC-4 or TLCC-5 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted TLCC-4 or TLCC-5 expression or activity. For example, such methods can be used to determine whether a subject can be effectively treated with an agent for a CNS disorder, pain disorder, or a cellular proliferation, growth, differentiation, or migration disorder. Thus, the present invention provides methods for determining whether a subject can be effectively treated with an agent for a disorder associated with aberrant or unwanted TLCC-4 or TLCC-5 expression or activity in which a test sample is obtained and TLCC-4 or TLCC-5 polypeptide or nucleic acid expression or activity is detected (e.g., wherein the abundance of TLCC-4 or TLCC-5 polypeptide or nucleic acid expression or activity is diagnostic for a subject that can be administered the agent to treat a disorder associated with aberrant or unwanted TLCC-4 or TLCC-5 expression or activity).

The methods of the invention can also be used to detect genetic alterations in a TLCC-4 or TLCC-5 gene, thereby determining if a subject with the altered gene is at risk for a disorder characterized by misregulation in TLCC-4 or TLCC-5 polypeptide activity or nucleic acid expression, such as a CNS disorder, pain disorder, or a disorder of cellular growth, differentiation, or migration. In preferred embodiments, the methods include detecting, in a sample of cells from the subject, the presence or absence of a genetic alteration characterized by at least one of an alteration affecting the integrity of a gene encoding a TLCC-4 or TLCC-5-polypeptide, or the mis-expression of the TLCC-4 or TLCC-5 gene. For example, such genetic alterations can be detected by ascertaining the existence of at least one of the following: 1) a deletion of one or more nucleotides from a TLCC-4 or TLCC-5 gene; 2) an addition of one or more nucleotides to a TLCC-4 or TLCC-5 gene; 3) a substitution of one or more nucleotides of a TLCC-4 or TLCC-5 gene; 4) a chromosomal rearrangement of a TLCC-4 or TLCC-5 gene; 5) an alteration in the level of a messenger RNA transcript of a TLCC-4 or TLCC-5 gene; 6) aberrant modification of a TLCC-4 or TLCC-5 gene, such as of the methylation pattern of the genomic DNA; 7) the presence of a non-wild type splicing pattern of a messenger RNA transcript of a TLCC-4 or TLCC-5 gene; 8) a non-wild type level of a TLCC-4 or TLCC-5 polypeptide; 9) allelic loss of a TLCC-4 or TLCC-5 gene; and 10) inappropriate post-translational modification of a TLCC-4 or TLCC-5 polypeptide. As described herein, there are a large number of assays known in the art which can be used for detecting alterations in a TLCC-4 or TLCC-5 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the TLCC-4 or TLCC-5 gene (see, e.g., Abravaya et al. (1995) Nucleic Acids Res .23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a TLCC-4 or TLCC-5 gene under conditions such that hybridization and amplification of the TLCC-4 or TLCC-5 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TLCC-4 or TLCC-5 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in TLCC-4 or TLCC-5 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in TLCC-4 or TLCC-5 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TLCC-4 or TLCC-5 gene and detect mutations by comparing the sequence of the sample TLCC-4 or TLCC-5 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the TLCC-4 or TLCC-5 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type TLCC-4 or TLCC-5 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation (see, for example, Cotton et a. (1988) Proc. Natl Acad Sci USA 85:4397; Saleeba et al. (1992) Methods Enzymol. 217:286-295). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TLCC-4 or TLCC-5 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a TLCC-4 or TLCC-5 sequence, e.g., a wild-type TLCC-4 or TLCC-5 sequence, is hybridized to a cDNA or other DNA product from a test cell(s). The duplex is treated with a DNA mismatch repair enzyme, and the cleavage products, if any, can be detected from electrophoresis protocols or the like (see, for example, U.S. Pat. No. 5,459,039).

In other embodiments, alterations in electrophoretic mobility will be used to identify mutations in TLCC-4 or TLCC-5 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal Tech. Appl. 9:73-79). Single-stranded DNA fragments of sample and control TLCC-4 or TLCC-5 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TLCC-4 or TLCC-5 gene.

Furthermore, any cell type or tissue in which TLCC-4 or TLCC-5 is expressed may be utilized in the prognostic assays described herein.

3. Monitoring of Effects During Clinical Trials

Monitoring the influence of agents (e.g., drugs) on the expression or activity of a TLCC-4 or TLCC-5 polypeptide (e.g., the modulation of membrane excitability) can be applied not only in basic drug screening, but also in clinical trials. For example, the effectiveness of an agent determined by a screening assay as described herein to increase TLCC-4 or TLCC-5 gene expression, polypeptide levels, or upregulate TLCC-4 or TLCC-5 activity, can be monitored in clinical trials of subjects exhibiting decreased TLCC-4 or TLCC-5 gene expression, polypeptide levels, or downregulated TLCC-4 or TLCC-5 activity. Alternatively, the effectiveness of an agent determined by a screening assay to decrease TLCC-4 or TLCC-5 gene expression, polypeptide levels, or downregulate TLCC-4 or TLCC-5 activity, can be monitored in clinical trials of subjects exhibiting increased TLCC-4 or TLCC-5 gene expression, polypeptide levels, or upregulated TLCC-4 or TLCC-5 activity. In such clinical trials, the expression or activity of a TLCC-4 or TLCC-5 gene, and preferably, other genes that have been implicated in, for example, a TLCC-4-associated disorder or TLCC-5-associated disorder can be used as a “read out” or markers of the phenotype of a particular cell.

For example, and not by way of limitation, genes, including TLCC-4 or TLCC-5, that are modulated in cells by treatment with an agent (e.g., compound, drug or small molecule) which modulates TLCC-4 or TLCC-5 activity (e.g., identified in a screening assay as described herein) can be identified. Thus, to study the effect of agents on TLCC-4-associated disorders or TLCC-5-associated disorders (e.g., disorders characterized by deregulated signaling or membrane excitation), for example, in a clinical trial, cells can be isolated and RNA prepared and analyzed for the levels of expression of TLCC-4 or TLCC-5 and other genes implicated in the TLCC-4-associated disorder or TLCC-5-associated disorder, respectively. The levels of gene expression (e.g., a gene expression pattern) can be quantified by northern blot analysis or RT-PCR, as described herein, or alternatively by measuring the amount of polypeptide produced, by one of the methods as described herein, or by measuring the levels of activity of TLCC-4 or TLCC-5 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a TLCC-4 or TLCC-5 polypeptide, mRNA, or genomic DNA in the preadministration sample; (iii) obtaining one or more post-administration samples from the subject; (iv) detecting the level of expression or activity of the TLCC-4 or TLCC-5 polypeptide, mRNA, or genomic DNA in the post-administration samples; (v) comparing the level of expression or activity of the TLCC-4 or TLCC-5 polypeptide, mRNA, or genomic DNA in the pre-administration sample with the TLCC-4 or TLCC-5 polypeptide, mRNA, or genomic DNA in the post administration sample or samples; and (vi) altering the administration of the agent to the subject accordingly. For example, increased administration of the agent may be desirable to increase the expression or activity of TLCC-4 or TLCC-5 to higher levels than detected, i.e., to increase the effectiveness of the agent. Alternatively, decreased administration of the agent may be desirable to decrease expression or activity of TLCC-4 or TLCC-5 to lower levels than detected, i.e. to decrease the effectiveness of the agent. According to such an embodiment, TLCC-4 or TLCC-5 expression or activity may be used as an indicator of the effectiveness of an agent, even in the absence of an observable phenotypic response.

4. Electronic Apparatus Readable Media and Arrays

Electronic apparatus readable media comprising TLCC-4 or TLCC-5 sequence information is also provided. As used herein, “TLCC-4 or TLCC-5 sequence information” refers to any nucleotide and/or amino acid sequence information particular to the TLCC-4 or TLCC-5 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said TLCC-4 or TLCC-5 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon TLCC-4 or TLCC-5 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the TLCC-4 or TLCC-5 sequence information.

A variety of software programs and formats can be used to store the sequence information on the electronic apparatus readable medium. For example, the sequence information can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the TLCC-4 or TLCC-5 sequence information.

By providing TLCC-4 or TLCC-5 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a TLCC-4 or TLCC-5-associated disease or disorder or a pre-disposition to a TLCC-4 or TLCC-5-associated disease or disorder, wherein the method comprises the steps of determining TLCC-4 or TLCC-5 sequence information associated with the subject and based on the TLCC-4 or TLCC-5 sequence information, determining whether the subject has a TLCC-4 or TLCC-5-associated disease or disorder or a pre-disposition to a TLCC-4 or TLCC-5-associated disease or disorder and/or recommending a particular treatment for the disease, disorder or pre-disease condition.

The present invention further provides in an electronic system and/or in a network, a method for determining whether a subject has a TLCC-4 or TLCC-5-associated disease or disorder or a pre-disposition to a disease associated with a TLCC-4 or TLCC-5 wherein the method comprises the steps of determining TLCC-4 or TLCC-5 sequence information associated with the subject, and based on the TLCC-4 or TLCC-5 sequence information, determining whether the subject has a TLCC-4 or TLCC-5-associated disease or disorder or a pre-disposition to a TLCC-4 or TLCC-5-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a TLCC-4 or TLCC-5-associated disease or disorder or a pre-disposition to a TLCC-4 or TLCC-5-associated disease or disorder associated with TLCC-4 or TLCC-5, said method comprising the steps of receiving TLCC-4 or TLCC-5 sequence information from the subject and/or information related thereto, receiving phenotypic information associated with the subject, acquiring information from the network corresponding to TLCC-4 or TLCC-5 and/or a TLCC-4 or TLCC-5-associated disease or disorder, and based on one or more of the phenotypic information, the TLCC-4 or TLCC-5 information (e.g., sequence information and/or information related thereto), and the acquired information, determining whether the subject has a TLCC-4 or TLCC-5-associated disease or disorder or a pre-disposition to a TLCC-4 or TLCC-5-associated disease or disorder. The method may further comprise the step of recommending a particular treatment for the disease, disorder or pre-disease condition.

The present invention also provides a business method for determining whether a subject has a TLCC-4 or TLCC-5-associated disease or disorder or a pre-disposition to a TLCC-4 or TLCC-5-associated disease or disorder, said method comprising the steps of receiving information related to TLCC-4 or TLCC-5 (e.g., sequence information and/or information related thereto), receiving phenotypic information associated with the subject, acquiring information from the network related to TLCC-4 or TLCC-5 and/or related to a TLCC-4 or TLCC-5-associated disease or disorder, and based on one or more of the phenotypic information, the TLCC-4 or TLCC-5 information, and the acquired information, determining whether the subject has a TLCC-4 or TLCC-5-associated disease or disorder or a pre-disposition to a TLCC-4 or TLCC-5-associated disease or disorder. The method may further comprise the step of recommending a particular treatment for the disease, disorder or pre-disease condition.

The invention also includes an array comprising a TLCC-4 or TLCC-5 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be TLCC-4 or TLCC-5. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a TLCC-4 or TLCC-5-associated disease or disorder, progression of TLCC-4 or TLCC-5-associated disease or disorder, and processes, such a cellular transformation associated with the TLCC-4 or TLCC-5-associated disease or disorder.

The array is also useful for ascertaining the effect of the expression of a gene on the expression of other genes in the same cell or in different cells (e.g., ascertaining the effect of TLCC-4 or TLCC-5 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including TLCC-4 or TLCC-5) that could serve as a molecular target for diagnosis or therapeutic intervention.

D. Methods of Treatment

The present invention provides for both prophylactic and therapeutic methods of treating a subject at risk of (or susceptible to) a disorder or having a disorder associated with aberrant or unwanted TLCC-4 or TLCC-5 expression or activity, e.g. a CNS disorder, pain disorder, or a cellular proliferation, growth, differentiation, or migration disorder. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the TLCC-4 or TLCC-5 molecules of the present invention or TLCC-4 or TLCC-5 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

A therapeutic agent includes, but is not limited to, small molecules, peptides, antibodies, ribozymes and antisense oligonucleotides.

1. Prophylactic Methods

In one aspect, the invention provides a method for preventing in a subject, a disease or condition associated with an aberrant or unwanted TLCC-4 or TLCC-5 expression or activity, by administering to the subject a TLCC-4 or TLCC-5 or an agent which modulates TLCC-4 or TLCC-5 expression or at least one TLCC-4 or TLCC-5 activity. Subjects at risk for a disease which is caused or contributed to by aberrant or unwanted TLCC-4 or TLCC-5 expression or activity can be identified by, for example, any or a combination of diagnostic or prognostic assays as described herein. Administration of a prophylactic agent can occur prior to the manifestation of symptoms characteristic of the TLCC-4 or TLCC-5 aberrancy, such that a disease or disorder is prevented or, alternatively, delayed in its progression. Depending on the type of TLCC-4 or TLCC-5 aberrancy, for example, a TLCC-4 or TLCC-5 agonist, or TLCC-4 or TLCC-5 antagonist agent can be used for treating the subject. The appropriate agent can be determined based on screening assays described herein.

2. Therapeutic Methods

Another aspect of the invention pertains to methods of modulating TLCC-4 or TLCC-5 expression or activity for therapeutic purposes. Accordingly, in an exemplary embodiment, the modulatory method of the invention involves contacting a cell capable of expressing TLCC-4 or TLCC-5 with an agent that modulates one or more of the activities of TLCC-4 or TLCC-5 polypeptide activity associated with the cell, such that TLCC-4 or TLCC-5 activity in the cell is modulated. An agent that modulates TLCC-4 or TLCC-5 polypeptide activity can be an agent as described herein, such as a nucleic acid or a polypeptide, a naturally-occurring target molecule of a TLCC-4 or TLCC-5 polypeptide (e.g., a TLCC-4 or TLCC-5 substrate), a TLCC-4 or TLCC-5 antibody, a TLCC-4 or TLCC-5 agonist or antagonist, a peptidomimetic of a TLCC-4 or TLCC-5 agonist or antagonist, or other small molecule. In one embodiment, the agent stimulates one or more TLCC-4 or TLCC-5 activities. Examples of such stimulatory agents include active TLCC-4 or TLCC-5 polypeptide and a nucleic acid molecule encoding TLCC-4 or TLCC-5 that has been introduced into the cell. In another embodiment, the agent inhibits one or more TLCC-4 or TLCC-5 activities. Examples of such inhibitory agents include antisense TLCC-4 or TLCC-5 nucleic acid molecules, anti-TLCC-4 or anti-TLCC-5 antibodies, and TLCC-4 or TLCC-5 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a TLCC-4 or TLCC-5 polypeptide or nucleic acid molecule. In one embodiment, the method involves administering an agent (e.g., an agent identified by a screening assay described herein), or combination of agents that modulates (e.g., upregulates or downregulates) TLCC-4 or TLCC-5 expression or activity. In another embodiment, the method involves administering a TLCC-4 or TLCC-5 polypeptide or nucleic acid molecule as therapy to compensate for reduced, aberrant, or unwanted TLCC-4 or TLCC-5 expression or activity.

Stimulation of TLCC-4 or TLCC-5 activity is desirable in situations in which TLCC-4 or TLCC-5 is abnormally downregulated and/or in which increased TLCC-4 or TLCC-5 activity is likely to have a beneficial effect. Likewise, inhibition of TLCC-4 or TLCC-5 activity is desirable in situations in which TLCC-4 or TLCC-5 is abnormally upregulated and/or in which decreased TLCC-4 or TLCC-5 activity is likely to have a beneficial effect.

3. Pharmacogenomics

The TLCC-4 or TLCC-5 molecules of the present invention, as well as agents, or modulators which have a stimulatory or inhibitory effect on TLCC-4 or TLCC-5 activity (e.g., TLCC-4 or TLCC-5 gene expression) as identified by a screening assay described herein can be administered to individuals to treat (prophylactically or therapeutically) TLCC-4-associated disorders or TLCC-5-associated disorders (e.g., proliferative disorders) associated with aberrant or unwanted TLCC-4 or TLCC-5 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a TLCC-4 or TLCC-5 molecule or TLCC-4 or TLCC-5 modulator as well as tailoring the dosage and/or therapeutic regimen of treatment with a TLCC-4 or TLCC-5 molecule or TLCC-4 or TLCC-5 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a TLCC-4 or TLCC-5 polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a TLCC-4 or TLCC-5 molecule or TLCC-4 or TLCC-5 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a TLCC-4 or TLCC-5 molecule or TLCC-4 or TLCC-5 modulator, such as a modulator identified by one of the exemplary screening assays described herein.

4. Use of TLCC-4 and TLCC-5 Molecules as Surrogate Markers

The TLCC-4 or TLCC-5 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the TLCC-4 or TLCC-5 molecules of the invention may be detected, and may be correlated with one or more biological states in vivo. For example, the TLCC-4 or TLCC-5 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The TLCC-4 or TLCC-5 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a TLCC-4 or TLCC-5 marker) transcription or expression, the amplified marker may be in a quantity which is more readily detectable than the drug itself. Also, the marker may be more easily detected due to the nature of the marker itself; for example, using the methods described herein, anti-TLCC-4 or anti-TLCC-5 antibodies may be employed in an immune-based detection system for a TLCC-4 or TLCC-5 protein marker, or TLCC-4-specific or TLCC-5-specific radiolabeled probes may be used to detect a TLCC-4 or TLCC-5 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20. The TLCC-4 or TLCC-5 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., TLCC-4 or TLCC-5 protein or RNA) for specific tumor markers in a subject, a drug or course of treatment may be selected that is optimized for the treatment of the specific tumor likely to be present in the subject. Similarly, the presence or absence of a specific sequence mutation in TLCC-4 or TLCC-5 DNA may correlate TLCC-4 or TLCC-5 drug response. The use of pharmacogenomic markers therefore permits the application of the most appropriate treatment for each subject without having to administer the therapy.

This invention is further illustrated by the following examples which should not be construed as limiting. The contents of all references, patents and published patent applications cited throughout this application, as well as the Figures and the Sequence Listing, are incorporated herein by reference.

### EXAMPLES

### EXAMPLE 1:

### Identification and Characterization of Human TLCC-4 and TLCC-5 cDNA

In this example, the identification and characterization of the genes encoding human TLCC-4 (clone Fbh48000) and TLCC-5 (clone Fbh52920c) is described.

### Isolation of the TLCC-4 and TLCC-5 cDNA

The invention is based, at least in part, on the discovery of human genes encoding novel polypeptides, referred to herein as TLCC-4 and TLCC-5. The entire sequence of the human clone Fbh48000 was determined and found to contain an open reading frame termed human “TLCC-4.” The nucleotide sequence of the human TLCC-4 gene is set forth in FIG. 1 and in the Sequence Listing as SEQ ID NO:1. The amino acid sequence of the human TLCC-4 expression product is set forth in FIG. 1 and in the Sequence Listing as SEQ ID NO:2. The TLCC-4 polypeptide encoded by this nucleic acid comprises about 751 amino acids. Clone Fbh48000, comprising the coding region of human TLCC-4, was deposited with the American Type Culture Collection (ATCC®), 10801 University Boulevard, Manassas, Va. 20110-2209, on______, and assigned Accession No.______.

The entire sequence of the human clone Fbh52920c was determined and found to contain an open reading frame termed human “TLCC-5.” The nucleotide sequence of the human TLCC-5 gene is set forth in FIG. 2 and in the Sequence Listing as SEQ ID NO:4. The amino acid sequence of the human TLCC-4 expression product is set forth in FIG. 2 and in the Sequence Listing as SEQ ID NO:5. The TLCC-5 polypeptide comprises about 1013 amino acids. Clone Fbh48000, comprising the coding region of human TLCC-5, was deposited with the American Type Culture Collection (ATCC®), 10801 University Boulevard, Manassas, Va. 20110-2209, on______, and assigned Accession No.______.

### Analysis of the Human TLCC-4 and TLCC-5 Molecules

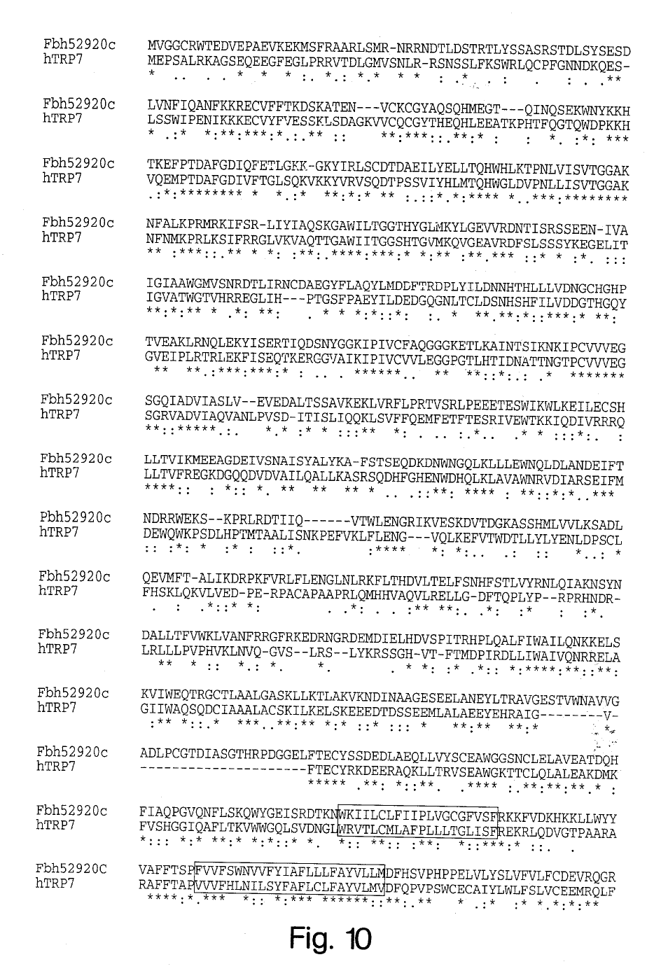

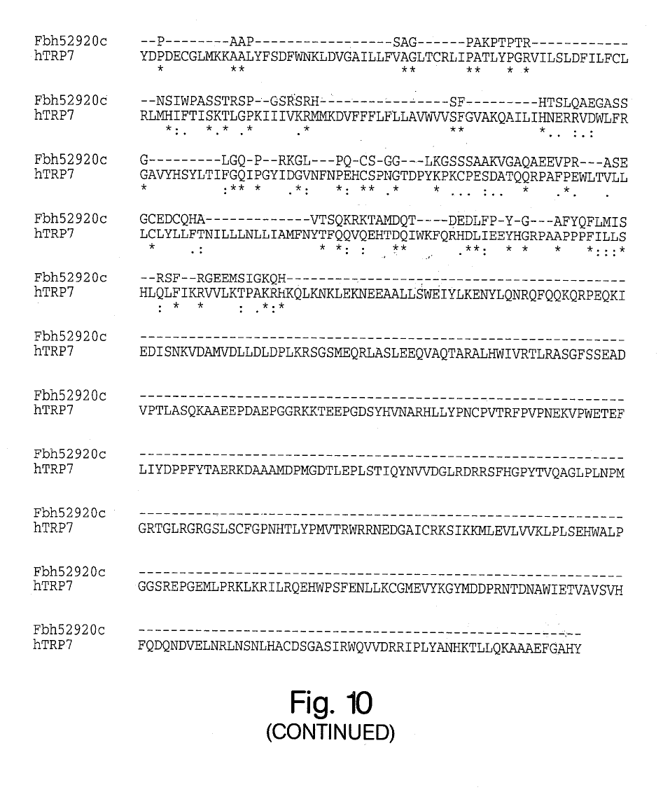

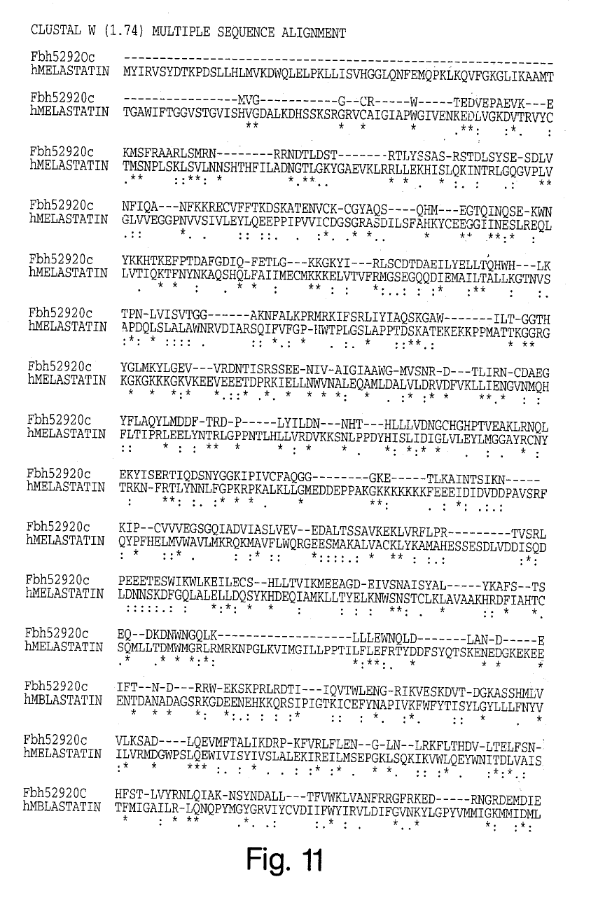

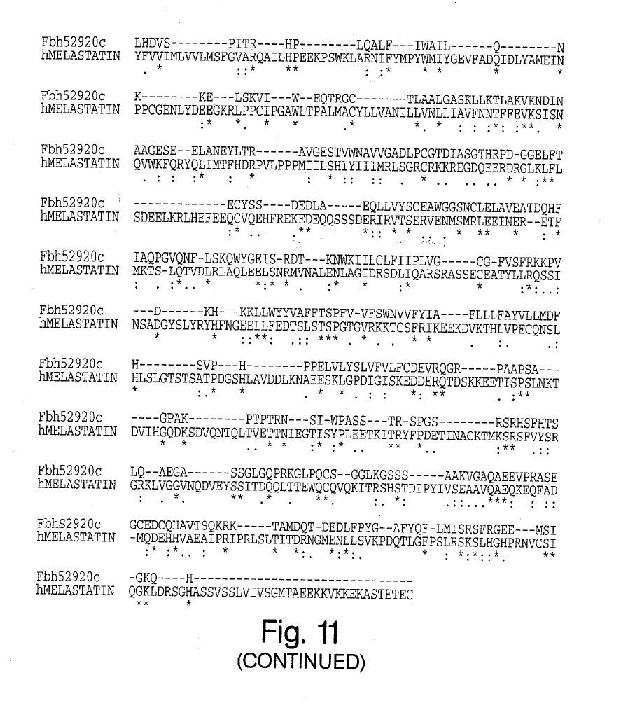

The human TLCC-5 amino acid sequence was aligned with the amino acid sequence of transient receptor potential polypeptide 7 (TRP7) and melastatin from Homo sapiens using the CLUSTAL W (1.74) multiple sequence alignment program. The results of the alignments are set forth in FIGS. 10 and 11.

A search was performed against the HMM database in PFAM (FIG. 5) resulting in the identification of four ankyrin repeat domains at about residues 167-202 (score=1.6), 214-246 (score=30.6), 261-294 (score=27.9), and 340-372 (score=18.6), and transport protein domain at about residues 510-677 (score=34.5) in the amino acid sequence of human TLCC-4 (SEQ ID NO:2).

A search was also performed against the MEMSAT database resulting in the identification of six transmembrane domains in the amino acid sequence of human TLCC-4 (SEQ ID NO:2) at about residues 440-461, 488-508, 520-540, 547-565, 590-609, and 652-676 (FIG. 6).

A search was further performed against the HMM database resulting in the identification of two transient receptor domains at about residues 720-778 (score=21.7) and 820-876 (score=1.5), in the amino acid sequence of human TLCC-5 (SEQ ID NO:5) (FIG. 8).

A search was also performed against the MEMSAT database resulting in the identification of two transmembrane domains in the amino acid sequence of human TLCC-5 (SEQ ID NO:5) at about residues 786-803 and 826-848 (FIG. 9).

A search in the Prosite database further resulted in the identification of several protein kinase C phosphorylation sites in the amino acid sequence of human TLCC-4 (SEQ ID NO:2) at about residues 37-39, 167-169, 290-292, 335-337, 374-376, 476-478, 498-500, and 688-690; several N-glycosylation sites in the amino acid sequence of human TLCC-4 (SEQ ID NO:2) at about residues 452-455 and 683-686; a cAMP- and cGMP-dependent protein kinase phosphorylation site in the amino acid sequence of human TLCC-4 (SEQ ID NO:2) at about residues 375-378; several casein kinase II phosphorylation sites in the amino acid sequence of human TLCC-4 (SEQ ID NO:2) at about residues 88-91, 163-166, 290-293, 305-308, 312-315, 388-391, 393-396, 397-400, 402-405, 411-414, 498-501, 607-610, 624-627, and 699-702; several tyrosine kinase phosphorylation sites in the amino acid sequence of human TLCC-4 (SEQ ID NO:2) at about residues 253-260, 375-382, and 614-622; several N-myristoylation sites in the amino acid sequence of human TLCC-4 (SEQ ID NO:2) at about residues 238-243 and 602-607; an amidation site in the amino acid sequence of human TLCC-4 (SEQ ID NO:2) at about residues 12-15; and a leucine zipper site in the amino acid sequence of human TLCC-4 (SEQ ID NO:2) at about residues 584-605.

A search performed in the Prosite database further resulted in the identification of several protein kinase C phosphorylation sites in the amino acid sequence of human TLCC-5 (SEQ ID NO:5) at about residues 21-23, 28-30, 39-41, 105-107, 240-242, 305-307, 331-333, 338-340, 711-713, 802-804, 901-903, 972-974, and 1001-1003; several casein kinase II phosphorylation sites in the amino acid sequence of human TLCC-5 (SEQ ID NO:5) at about residues 54-57, 143-146, 223-226, 240-243, 308-311, 360-363, 436-439, 487-490, 576-579, 725-728, 977-980, and 982-985; and several tyrosine kinase phosphorylation sites in the amino acid sequence of human TLCC-5 (SEQ ID NO:5) at about residues 49-55, 247-254, and 307-314.

Further domain motifs were identified by using the amino acid sequence of TLCC-4 (SEQ ID NO:2) to search through the ProDom database. Numerous matches against protein domains described as “receptor vanilloid channel activated receptor-related receptor-like type OTRPC4”, “channel vanilloid receptor activated receptor-related receptor-like OTRPC4 2B ion”, “repeat ankyrin kinase nuclear factor channel”, “ankyrin repeat kinase domain UNC-44 alternative glycoprotein EGF-like”, “ankyrin”, “channel osmotically receptor-related vanilloid cation”, “receptor vanilloid channel activated receptor-related receptor-like calcium type”, “calcium epithelial channel transporter homolog CAT2”, “channel protein receptor calcium transient potential transmembrane ion transport”, and “receptor vanilloid channel activated osmotically”, and the like were identified.

Further domain motifs were identified by using the amino acid sequence of TLCC-5 (SEQ ID NO:5) to search through the ProDom database. Numerous matches against protein domains described as “channel protein calcium entry capacitative ionic transmembrane ion transport transient” and the like were identified.

### Tissue Distribution of TLCC-4 and TLCC-5 mRNA

This example describes the tissue distribution of TLCC-4 mRNA, as determined by RT-PCR, and the tissue distribution of TLCC-4 and TLCC-5 mRNA as may be determined by Northern blot analysis.

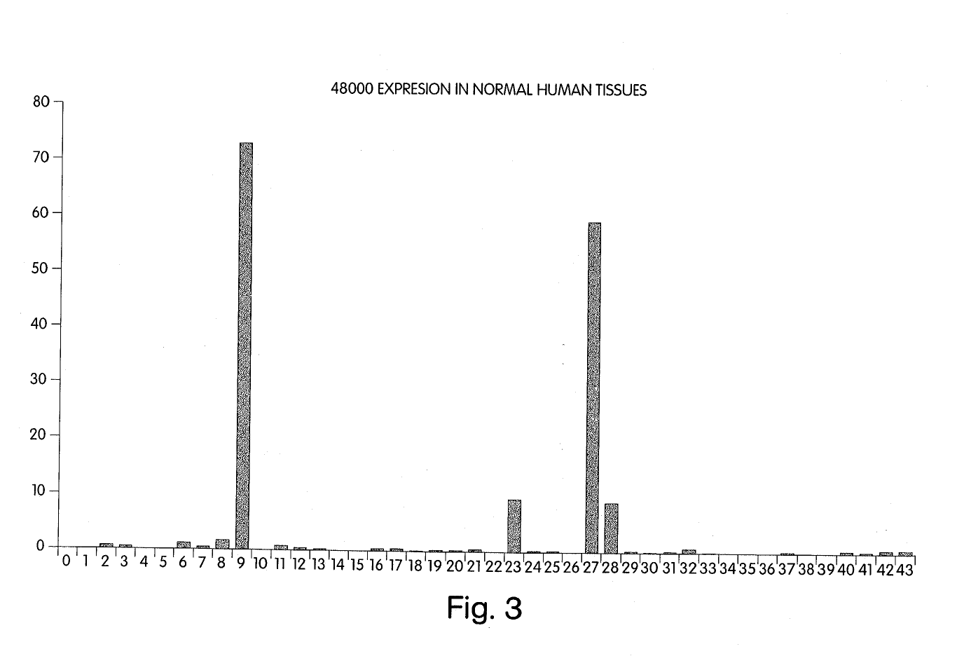

Various cDNA libraries were analyzed by RT-PCR using a human TLCC-4-specific probe. From this analysis it was determined that TLCC-4 mRNA was expressed predominantly in the hypothalamus and skin. TLCC-4 mRNA was found in moderate levels in adipose and teste, and in low levels in skeletal muscle and brain (see FIG. 3). Northern blot hybridizations with the various RNA samples is performed under standard conditions and washed under stringent conditions, i.e., 0.2×SSC at 65° C. The DNA probe is radioactively labeled with ³²P-dCTP (using the Prime-It kit (Stratagene, La Jolla, Calif.) according to the instructions of the supplier). Filters containing human tissue mRNA (MultiTissue Northern I and MultiTissue Northern II from Clontech, Palo Alto, Calif.) are probed in ExpressHyb hybridization solution (Clontech) and washed at high stringency according to manufacturer's recommendations.

In situ hybridization experiments were performed using a human TLCC-4-specific probe indicating TLCC-4 expression in monkey brain (cortex, thalamus, caudate, and hippocampus), spinal cord, DRG and SRG neurons, and in hair follicles. In situ hybridization with rat pain models indicated that TLCC-4 mRNA was down-regulated after chronic constriction injury, which causes persistent, spontaneous firing of neurons and results in pain. TLCC-4 mRNA was also down-regulated after treatment with clofibric acid, a selective muscle toxin which produces muscle pain and inflammation.

### EXAMPLE 2

### Expression of Recombinant TLCC-4 and TLCC-5 Polypeptides in Bacterial Cells

In this example, human TLCC-4 or human TLCC-5 is expressed as a recombinant glutathione-S-transferase (GST) fusion polypeptide in E. coli and the fusion polypeptide is isolated and characterized. Specifically, TLCC-4 or TLCC-5 is fused to GST and this fusion polypeptide is expressed in E. coli, e.g., strain PEB199. Expression of the GST-TLCC-4 or GST-TLCC-5 fusion protein in PEB 199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### EXAMPLE 3

### Expression of Recombinant IC54420 Polypeptide in COS Cells

To express the human TLCC-4 or human TLCC-5 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire TLCC-4 or TLCC-5 polypeptide and an HA tag (Wilson et al. (1984) Cell 37:767) or a FLAG tag fused in-frame to its 3′ end of the fragment is cloned into the polylinker region of the vector, thereby placing the expression of the recombinant polypeptide under the control of the CMV promoter.

To construct the plasmid, the human TLCC-4 or human TLCC-5 DNA sequence is amplified by PCR using two primers. The 5′ primer contains the restriction site of interest followed by approximately twenty nucleotides of the TLCC-4 or TLCC-5 coding sequence starting from the initiation codon; the 3′ end sequence contains complementary sequences to the other restriction site of interest, a translation stop codon, the HA tag or FLAG tag and the last 20 nucleotides of the TLCC-4 or TLCC-5 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the TLCC-4 or TLCC-5 gene is inserted in the correct orientation. The ligation mixture is transformed into E. coli cells (strains HB 101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the human TLCC-4- pcDNA/Amp plasmid DNA or human TLCC-5-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the IC54420 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the human TLCC-4 or human TLCC-5 coding sequence is cloned directly into the polylinker of the pCDNA/Amp vector using the appropriate restriction sites. The resulting plasmid is transfected into COS cells in the manner described above, and the expression of the TLCC-4 or TLCC-5 polypeptide is detected by radiolabelling and immunoprecipitation using a TLCC-4-specific or TLCC-5-specific monoclonal antibody.

### EXAMPLE 4

### Tissue Distribution of Human TLCC-4 mRNA Using Taqman™ Analysis

This example describes the tissue distribution of human TLCC-4 mRNA in a variety of cells and tissues, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., various human tissue samples, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′, N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

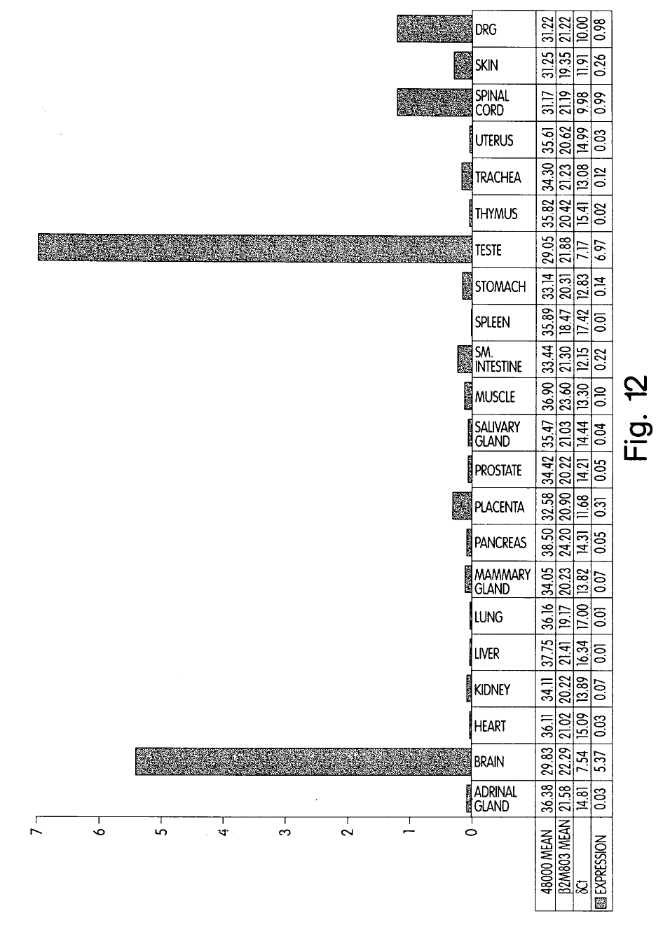

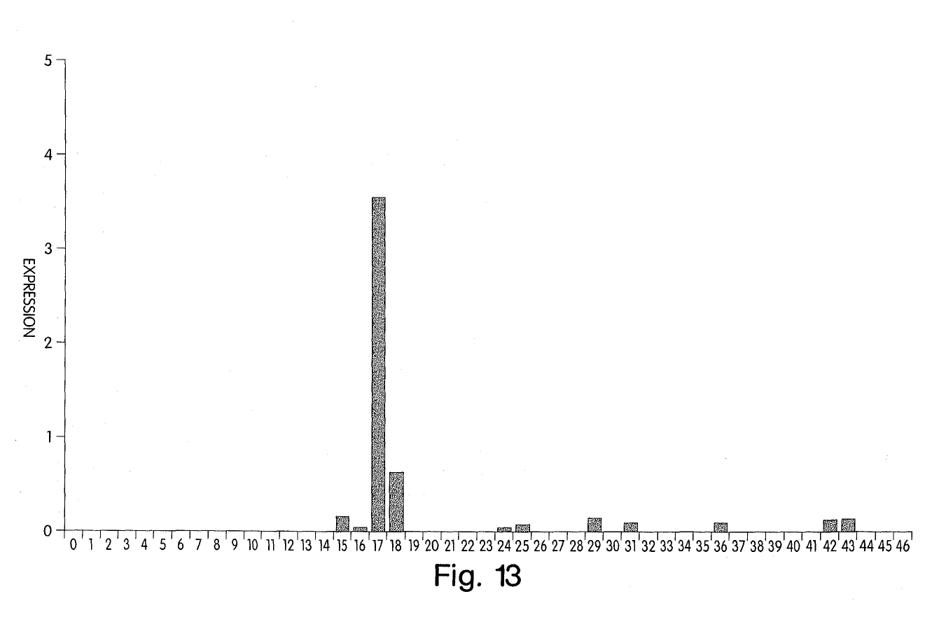

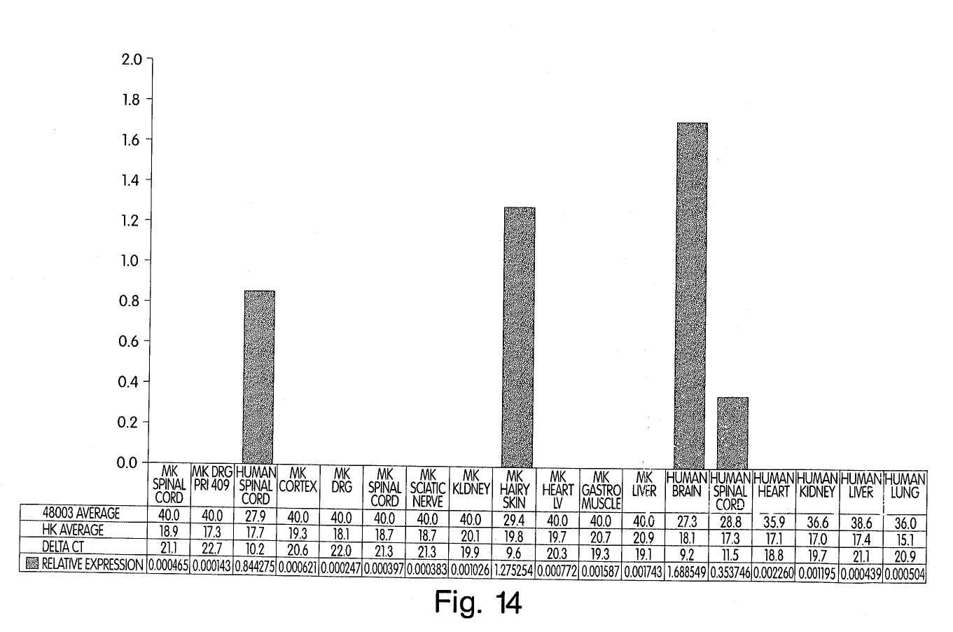

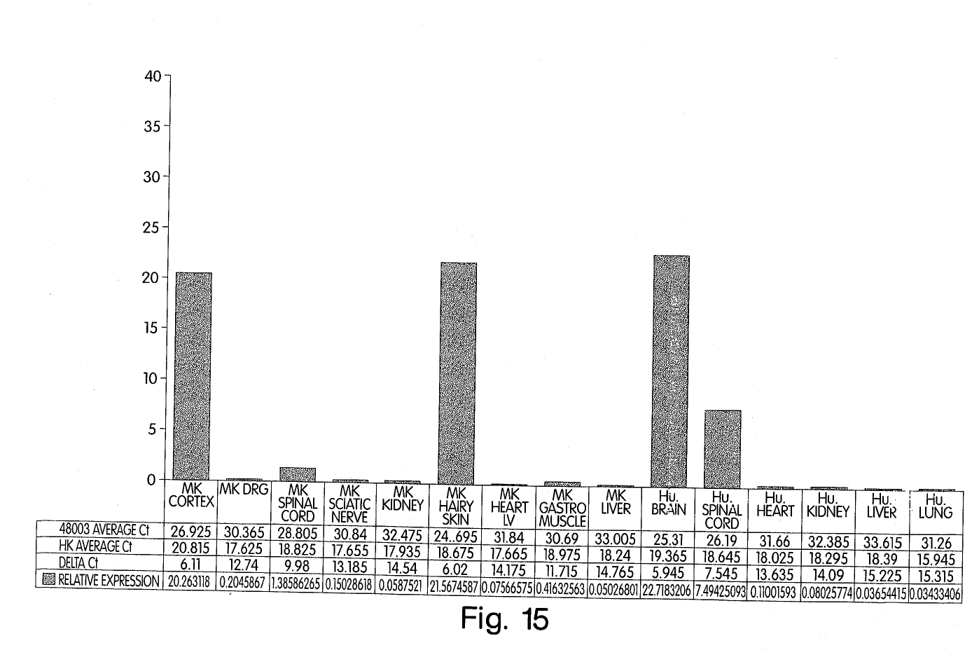

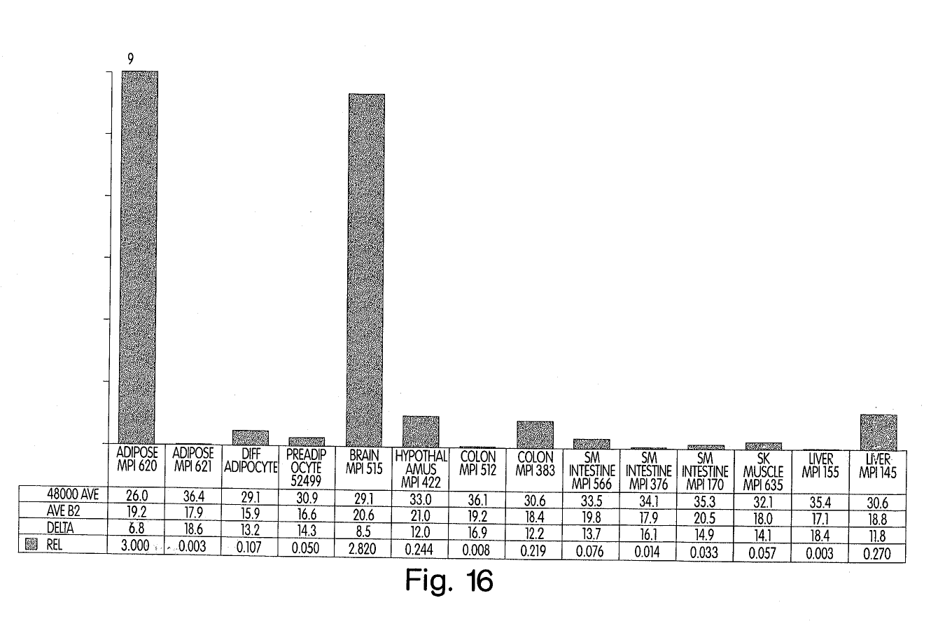

As indicated in FIG. 3, strong expression of TLCC-4 was detected in human brain (hypothalamus) and skin tissues. In addition, TLCC-4 expression was detected at moderate levels in adipose and testis tissues, and at low levels in the fetal heart, skeletal muscle, brain, and colon tissues. Pain human panel phase I and MP Phase 1.3.3 libraries were also analyzed and it was determined that TLCC-4 was expressed at high levels in the brain, cortex, and testis, at moderate levels in the spinal cord, dorsal root ganglion (DRG), and the hypothalamus, and at low levels in the skin, placenta, small intestine, ovary, prostate epithelial cells, liver, skin (decubitus), colon tumor cells, and breast tumor cells (see FIGS. 12 and 13). Monkey libraries were also analyzed indicating that TLCC-4 was expressed at high levels in the monkey cortex and hairy skin, and at low levels in the monkey spinal cord (see FIGS. 14 and 15). Metabolic libraries were also analyzed demonstrating that TLCC-4 was expressed at high levels in adipose and brain tissues, and at low levels in differentiated adipocytes and pre-adipocytes, as well as in the hypothalamus, colon, small intestine, skeletal muscle, and liver tissues (see FIG. 16).

### EXAMPLE 5

### Regulation of Calcium Influx Through TLCC-4

This experiment describes the regulation of calcium influx though TLCC-4 in 911 cells as determined by Fluorometric Imaging Plate Reader experiments (FLIPR) (Molecular Devices Corp., Sunnyvale, Calif.).

The FLIPR is a screening tool for cell-based fluorescent assays which allows the simultaneous stimulation and measurement of separate cell populations in a high throughput format. Therefore, using this system, it is possible to quantify transient signals, such as the release of intracellular calcium, from cell populations, in parallel and in real time. The FLIPR contains chambers in which to hold the test plate and plates containing antagonists or agonists to be added to the test plate. The FLIPR utilizes an argon laser that provides discrete spectral lines spaced from approximately 350 to 530 nm. For use with fluorescent Ca²⁺dyes, the 88-nm line of the laser is employed. The laser simultaneously illuminates the wells in a test plate. The image of each well in the plate is captured by a cooled charge coupled device (CCD) camera, which updates images once per second, if required, for the measurement of rapid calcium responses. Because both excitation and emission are read via the bottom of the plate, black-walled, transparent bottomed 96-well plates are used. Data captured by the CCD camera is converted to digital data and then transferred to a computer.

Briefly, a calcium indicator (e.g., fluo-3/AM or Calcium Green-1/AM) was transferred to the culture medium. Because the FLIPR collects fluorescence from the bottom of the well, suspension cells require centrifugation to the base of the well following dye loading. Viable 911 cells were resuspended in loading medium and incubated for one hour. The cells were then centrifuged and resuspended with wash buffer. The cell suspension containing the dye was then aliquoted into each well of the black-walled, transparent bottomed 96-well plate and the plate was centrifuged. The FLIPR assay was then carried out and the results analyzed. (If adherent cells are used, they may be plated at an appropriate density in the 96-well plates and cultured overnight. Dye may then be loaded and incubated).

Results show a constitutive calcium influx through TLCC-4 in 911 cells that were incubated with NMDG/0 Ca²⁺ and stimulated afterwards with 5 mM Ca²⁺.

### Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
(a) a nucleic acid molecule comprising the nucleotide sequence set forth in SEQ ID NO:1; and
(b) a nucleic acid molecule comprising the nucleotide sequence set forth in SEQ ID NO:3; and
(c) a nucleic acid molecule comprising the nucleotide sequence set forth in SEQ ID NO:4; and
(d) a nucleic acid molecule comprising the nucleotide sequence set forth in SEQ ID NO:6.

2. An isolated nucleic acid molecule selected from the group which encodes:
(a) a polypeptide comprising the amino acid sequence set forth in SEQ ID NO:2; and
(b) a polypeptide comprising the amino acid sequence set forth in SEQ ID NO:5.

3. Isolated nucleic acid molecules comprising the nucleotide sequences contained in the plasmids deposited with ATCC® as Accession Numbers______.

4. An isolated nucleic acid molecule which encodes a naturally occurring allelic variant of a polypeptide comprising the amino acid sequence set forth in SEQ ID NO:2 or 5.

5. An isolated nucleic acid molecule selected from the group consisting of:
a) a nucleic acid molecule comprising a nucleotide sequence which is at least 60% identical to the nucleotide sequence of SEQ ID NO:1, 3, 4, or 6, or a complement thereof;
b) a nucleic acid molecule comprising a fragment of at least 30 nucleotides of a nucleic acid comprising the nucleotide sequence of SEQ ID NO:1, 3, 4, or 6, or a complement thereof,
c) a nucleic acid molecule which encodes a polypeptide comprising an amino acid sequence at least about 60% identical to the amino acid sequence of SEQ ID NO:2 or 5; and
d) a nucleic acid molecule which encodes a fragment of a polypeptide comprising the amino acid sequence of SEQ ID NO:2 or 5, wherein the fragment comprises at least 10 contiguous amino acid residues of the amino acid sequence of SEQ ID NO:2 or 5.

6. An isolated nucleic acid molecule which hybridizes to a complement of the nucleic acid molecule of any one of claims 1, 2, 3, 4, or 5 under stringent conditions.

7. An isolated nucleic acid molecule comprising a nucleotide sequence which is complementary to the nucleotide sequence of the nucleic acid molecule of any one of claims 1, 2, 3, 4, or 5.

8. An isolated nucleic acid molecule comprising the nucleic acid molecule of any one of claims 1, 2, 3, 4, or 5, and a nucleotide sequence encoding a heterologous polypeptide.

9. A vector comprising the nucleic acid molecule of any one of claims 1, 2, 3, 4, or 5.

10. The vector of claim 9, which is an expression vector.

11. A host cell transfected with the expression vector of claim 10.

12. A method of producing a polypeptide comprising culturing the host cell of claim 11 in an appropriate culture medium to, thereby, produce the polypeptide.

13. An isolated polypeptide selected from the group consisting of:
a) a fragment of a polypeptide comprising the amino acid sequence of SEQ ID NO:2 or 5, wherein the fragment comprises at least 10 contiguous amino acids of SEQ ID NO:2 or 5;
b) a naturally occurring allelic variant of a polypeptide comprising the amino acid sequence of SEQ ID NO:2 or 5, wherein the polypeptide is encoded by a nucleic acid molecule which hybridizes to a complement of a nucleic acid molecule consisting of SEQ ID NO:1, 3, 4, or 6 under stringent conditions;
c) a polypeptide which is encoded by a nucleic acid molecule comprising a nucleotide sequence which is at least 60% identical to a nucleic acid comprising the nucleotide sequence of SEQ ID NO:1, 3, 4, or 6; and
d) a polypeptide comprising an amino acid sequence which is at least 60% identical to the amino acid sequence of SEQ ID NO:2 or 5.

14. The isolated polypeptide of claim 13 comprising the amino acid sequence of SEQ ID NO:2 or 5.

15. The polypeptide of claim 13, further comprising heterologous amino acid sequences.

16. An antibody which selectively binds to a polypeptide of claim 13.

17. A method for detecting the presence of a polypeptide of claim 13 in a sample comprising:
a) contacting the sample with a compound which selectively binds to the polypeptide; and
b) determining whether the compound binds to the polypeptide in the sample to thereby detect the presence of a polypeptide of claim 13 in the sample.

18. The method of claim 17, wherein the compound which binds to the polypeptide is an antibody.

19. A kit comprising a compound which selectively binds to a polypeptide of claim 13 and instructions for use.

20. A method for detecting the presence of a nucleic acid molecule of any one of claims 1, 2, 3, 4, or 5 in a sample comprising:
a) contacting the sample with a nucleic acid probe or primer which selectively hybridizes to a complement of the nucleic acid molecule; and
b) determining whether the nucleic acid probe or primer binds to the complement of the nucleic acid molecule in the sample to thereby detect the presence of the nucleic acid molecule of any one of claims 1, 2, 3, 4, or 5 in the sample.

21. The method of claim 20, wherein the sample comprises mRNA molecules and is contacted with a nucleic acid probe.

22. A kit comprising a compound which selectively hybridizes to a complement of the nucleic acid molecule of any one of claims 1, 2, 3, 4, or 5 and instructions for use.

23. A method for identifying a compound which binds to a polypeptide of claim 13 comprising:
a) contacting the polypeptide, or a cell expressing the polypeptide with a test compound; and
b) determining whether the polypeptide binds to the test compound.

24. The method of claim 23, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for TLCC-4 or TLCC-5 activity.

25. A method for modulating the activity of a polypeptide of claim 13 comprising contacting the polypeptide or a cell expressing the polypeptide with a compound which binds to the polypeptide in a sufficient concentration to modulate the activity of the polypeptide.

26. A method for identifying a compound which modulates the activity of a polypeptide of claim 13 comprising:
a) contacting a polypeptide of claim 13 with a test compound; and
b) determining the effect of the test compound on the activity of the polypeptide to thereby identify a compound which modulates the activity of the polypeptide.

27. The method of claim 26, wherein said activity is modulation of nociception.

28. A method for identifying a compound which modulates pain comprising:
a) contacting the polypeptide of claim 13, or a cell expressing the polypeptide with a test compound; and
b) identifying the compound as a modulator of pain by determining the effect of the test compound on the activity of the polypeptide.

29. A method for identifying a compound which modulates nociception comprising:
a) contacting the polypeptide of claim 13, or a cell expressing the polypeptide with a test compound; and
b) identifying the compound as a modulator of nociception by determining the effect of the test compound on the activity of the polypeptide.

30. A method for treating a subject having a pain disorder comprising administering to the subject a TLCC-4 or TLCC-5 modulator, thereby treating said subject having a pain disorder.

31. A method for treating a subject having a pain disorder comprising administering to the subject a TLCC-4 or TLCC-5 modulator, wherein the TLCC-4 or TLCC-5 modulator is the modulator identified by the method of claim 26, thereby treating said subject having a pain disorder.

32. The method of claim 30, wherein the TLCC-4 or TLCC-5 modulator is a small molecule.

33. The method of claim 30, wherein said TLCC-4 or TLCC-5 modulator is administered in a pharmaceutically acceptable formulation.

34. The method of claim 30, wherein said TLCC-4 or TLCC-5 modulator is administered using a gene therapy vector .

35. The method of 30, wherein the TLCC-4 or TLCC-5 modulator is capable of modulating TLCC-4 or TLCC-5 polypeptide activity.
